(19) United States
(12) Patent Application Publication
(10) Pub. No.: US 2026/0060705 A1
(43) Pub. Date: Mar. 5, 2026

(54) TEMPORARY VASCULAR SCAFFOLD WITH ADJUNCTIVE ENERGY DELIVERY

(71) Applicant: Nfinium Vascular Technologies, LLC, Grand Junction, CO (US)

(72) Inventor: Richard Eustis FULTON, III, Grand Junction, CO (US)

(73) Assignee: Nfinium Vascular Technologies, LLC, Grand Junction, CO (US)

(21) Appl. No.: 19/379,760

(22) Filed: Nov. 5, 2025

Related U.S. Application Data
(63) Continuation of application No. PCT/US24/29317, filed on May 14, 2024.
(60) Provisional application No. 63/466,546, filed on May 15, 2023.

(51) Int. Cl. A61B 17/22; A61B 17/00; A61B 17/3207

(52) U.S. Cl. CPC A61B 17/22012; A61B 17/320725; A61B 2017/00557; A61B 2017/00778; A61B 2017/00893; A61B 2017/22002; A61B 2017/22025; A61B 2017/22065

(57) ABSTRACT

Devices and methods for treating a target site in a body lumen are provided. A medical device includes a stent-like structure including a plurality of scoring and non-scoring filaments interwoven with one another. Generally, the stent-like structure may have more non-scoring filaments than scoring filaments to provide greater structural support and to focus the scoring forces on only a few select areas. The stent-like structure is expanded within the target site to score the target site and to provide temporary structural support while the target site is infused with a therapeutic agent. Such therapeutic agent infusion occurs with the use of a drug eluting or drug coated balloon disposed within the stent-like structure or by occluding the target site and introducing a drug into the occluded target site to sit for a period of time.

### CROSS-REFERENCE

The present application is a continuation of PCT/US2024/029317, filed May 14, 2024, which claims the benefit of U.S. Provisional Application No. 63/466,546, filed May 15, 2023, the content of each of which are fully incorporated herein by reference.

### BACKGROUND OF THE INVENTION

Balloon angioplasty has been a popular method of treating vascular occlusions since 1976. With plain old balloon angioplasty (POBA), there exists a significant subset of patients who have immediate suboptimal results related to the trauma to the vessel including dissection of the vessel, incomplete plaque compression, poor lumen gain, and acute elastic recoil of the vessel, amongst others. Because of these suboptimal immediate results, which occur in 40-60% or more of cases, other means to treat vascular stenosis were developed. Intravascular stents are widely utilized, addressing the acute problems of angioplasty and reducing the restenosis rates from 50-60% for POBA to 30-35% for these bare metal stents (BMS).

Because the restenosis rates of BMS are usually unacceptable, drug eluting stents (DES) are used to inhibit restenosis. These devices reduce the restenosis rate to around 20% and lower in the coronary circulation. However, DES are extremely expensive and can lead to thrombosis, which can prove fatal. In addition, DES are not particularly effective in the peripheral circulation. The expense of drug eluting stents dramatically increases the overall cost of healthcare in the U.S. Finally, not only are the stents costly, but expensive and potentially harmful drugs are routinely used for at least a year after stent implantation.

Restenosis is an Achilles heel of all vascular intervention, from angioplasty to stenting and even surgery. Various drugs can prevent restenosis. A primary question is how best to deliver the drugs in the most cost effective manner available while producing good patient outcomes and preventing complications.

Because of variable plaque morphology and composition, stresses provided by conventional POBA can be unpredictable, and frequently high pressure balloon inflations are often needed to successfully provide enough stress to crack the plaque. When the plaque does compress at high pressures, the balloon will often very rapidly expand to its full dimension in a noticeable pop (tenths of a second) and very rapidly expanding the vessel wall, which can rupture the smooth muscle cells. Dissections frequently occur, as does irreparable injury to the smooth muscle cells which do not have the chance to gradually stretch and deform to maintain their integrity.

Many vessels are heavily calcified which inhibits compression and the expected lumen gain. The term “calcific matrix” as used henceforth, describes the compression resistant calcification whether it is within the arterial media, arterial intima, or the plaque, or all of these locations.

Therefore, methods and devices that lower the pressure at which the plaque or calcific matrix may fracture may produce a slower and more gradual stretching of the arterial wall. This slower stretching can diminish the degree of trauma to the vessel. Methods and devices which allow the slow, gradual expansion and a prolonged distension of the vessel wall may allow plastic deformation of the tissue to occur while distension is present and to prevent elastic recoil of the vessel.

A wire or wires along the outside of an angioplasty balloon, sometimes called buddy wires, can produce focal areas of stresses along the wires that are approximately 120 times that of a conventional balloon surface, and the stress patterns from the external wire can extend into the plaque rather than being concentrated on the surface as with a conventional balloon. The stress patterns are typically less dependent on the morphology and composition of the plaque than with conventional balloons. In other words, the stresses can be more predictable and concentrated and require lower balloon pressures to compress the plaque. Clinical studies have confirmed that when compared to conventional POBA catheters, the buddy wire technique compressed the plaque at lower balloon pressures, caused fewer dissections, had less elastic recoil, and had more lumen gain, as well as a trend toward lower restenosis rate.

Subsequently, cutting and scoring balloons have been introduced extending these concepts. One such balloon uses several razor type blades along the balloon margins. Scoring balloons may utilize several 0.005 to 0.007 inch struts placed over a balloon or other scoring elements placed on the surface of a balloon. Both balloon types are commercially successful. They are typically used in treating complex lesions or in plaque modification. The scoring balloon has been shown to achieve 50% more lumen gain than POBA when utilized as predilatation before stent implantation. This procedure can significantly reduce the number of dissections when compared to POBA. The scoring balloon also has been shown to not slip off of the lesion, which is a problem with POBA. The scoring balloon can also be more effective in soft, fibrous, and calcified plaques than POBA and has been recommended as a strategy of plaque modification in treating complex lesions. The use of the scoring balloon has thus resulted in very low incidences of inadvertent or unplanned stenting, commonly referred to as bail out stenting.

Slow, gradual balloon inflation and prolonged balloon inflation both improve the immediate results of POBA with fewer dissections, fewer further interventions such as stenting, and less restenosis. On the other hand, other studies did not show improvement in long term results with prolonged inflation times, possibly because their prolonged inflations were the result of treating dissections. Currently, no studies that evaluate both plaque modification and gradual inflation/prolonged distension have been conducted or published.

While these mechanical strategies have resulted in measurable improvement in the acute complications of POBA, a promising advancement in POBA has been the advent of drug coated balloons (DCB's). A DCB is a POBA balloon coated with an antiproliferative drug, such as paclitaxel or sirolimus. The drug is delivered during the rather short balloon inflation and has been shown to be present in smooth muscle cells up to six days later. The drug from a DCB covers essentially 100% of the plaque/vessel wall vs. only 15-20% with drug eluting stents. Use of DCB's has dramatically reduced the restenosis rate, at least in coronary and above the knee peripheral interventions.

Therefore, both mechanical and pharmacological strategies have shown advantages in treating vascular lesions with balloon angioplasty. The mechanical strategies effectively address the acute or immediate problems by causing less injury to the vessel and the pharmacological strategy of drug coated balloons significantly diminish restenosis.

In U.S. Pat. No. 5,449,372, Schmaltz et al. describe a temporary stent that can be used for support after dilatation of the lesion. In U.S. Pat. Nos. 6,450,989 and 7,011,654, William R. Dubrul and Richard E. Fulton describe a dilating and support apparatus with disease inhibitors and methods of use. In U.S. Pat. No. 7,232,432, William R. Dubrul and Richard E. Fulton describe a porous braided structure for angioplasty and drug delivery. The following U.S. Patents and Publications may also be of interest: U.S. Pat. Nos. 8,454,636, 7,494,497, 7,279,002, 6,808,531, 5,797,935, and 5,766,203, and U.S. Publication Nos. 2013/0041391, 2011/0082483, and 2005/0080478. Those patents and applications are incorporated herein by reference.

The current disclosure comprises new improvements to the prior inventions as well as novel methods of utilizing both the prior inventions and the new improvements to them.

### BRIEF SUMMARY OF THE INVENTION

To address the problem of how to treat a lesion in the most cost effective manner available while producing good patient outcomes and preventing complications, the medical device industry has essentially focused on developing methods and devices that inhibit the vascular response to the injury (restenosis), as opposed to developing a device that causes less injury, and hence less restenosis. One aspect of the present invention is directed to a device and method that both causes less injury to the vasculature by the use of dilatation of a specialized scaffold over a balloon. The device and method provided herein can cause less dissection and more even plaque disruption at lower pressures. The device and method provided herein can introduce drug deep within the vessel wall while providing means for prolonged vessel distension that will improve acute lumen gain and reduce vascular recoil. Thus less initial injury and less elastic recoil may result in less restenosis, and delivering a drug may further reduce or prevent the restenosis. Furthermore, while many of the above features have been described in previous patents, additional structural improvements and additions to the previously described art can be revealed along with novel methods of utilizing the prior art and the current improved inventions.

It is the immediate result of an intervention (the immediate lumen diameter and the immediate residual percent stenosis) that typically determines the late outcome after vascular interventions. Many embodiments of the present invention are designed to improve these two factors. An optimal outcome in percutaneous interventions may depend upon: 1) obtaining an excellent acute angiographic results with less intimal dehiscence, dissection, and elastic recoil; 2) avoiding damage to the distal vascular bed from procedural liberated debris (as compared to the liberated debris in atherectomy); and 3) reducing smooth muscle cell proliferation by causing less injury and by pharmacological intervention. Aspects of the present invention address all three areas.

Many embodiments of the present invention are directed to a method of treating a target site within a vascular channel of the body using a catheter assembly, the catheter assembly comprising a stent or scaffold like structure mounted over an expandable balloon. The method of using such device to perform an intervention at the target site can comprise inserting the device to the target site and expanding the expandable balloon element slowly and gradually over a time period of at least 1 minute, but preferably over 2-3 minutes to a pressure in the range of 5-6 atm. Expanding the expandable balloon can expand the scaffold against the wall to the desired diameter. The method can further comprise collapsing the balloon while the scaffold remains expanded for a prolonged period of time greater than 5 minutes. During this time, plastic deformation of the plaque and tissues occurs which can increase lumen gain and reduce vascular recoil. The method can further comprise collapsing the scaffold over the already collapsed balloon and removing the entire device.

In summary, the prior art method comprises expanding an expandable element, such as a balloon and a temporary stent structure covering the balloon, against an inner wall of the vascular channel. In some embodiments, the balloon is collapsed leaving the stent structure expanded against the inner wall for a period of time of at least three minutes, and the collapsed balloon and the collapsed stent structure are removed from the vascular channel during the stent structure removing step. The current improved method of treating a target site includes the steps of the prior art method plus one or more of a slow, incremental inflation of the expandable element over at least 90 seconds with pressure variation pulses added to the incremental inflation, scoring of the plaque/vessel with specialized scoring elements, delivery of an adjunctive energy source to cause structural instability in the calcific matrix, delivery of motion of the catheter or scaffold to weaken the calcific matrix, and rapid pressure changes to the vessel wall to weaken the calcific matrix and more completely compress the plaque and calcific matrix.

In many embodiments, the balloon stent assembly comprises a catheter assembly having a proximal portion and a distal portion. The catheter assembly may comprise a first and a second elongate members. A temporary stent (or scaffold as used interchangeably herein) may have proximal and distal ends; the proximal end being secured to a first position along the first elongate member and the distal end being secured to a second position along the second elongate member. The temporary stent may be placeable in a contracted state by movement of the first and second positions of the elongate members away from one another. The assembly may also include an inflatable balloon mounted to the distal portion of the catheter assembly at a location surrounded by the temporary stent. The balloon may be placeable in an inflated state, thereby placing the temporary stent in an expanded state, and in a collapsed state. The temporary stent can be free to remain in the expanded state when the balloon moves to the collapsed state. The temporary stent may comprise scoring members. Energy delivery means may be provided within the device or separate from the device configured to be inserted coaxially into the device. Means for providing motion to the catheter, guide wire, or scaffold may be provided. Means to rapidly alter the pressure upon the vessel may also be provided.

Alternatively, the scaffold (or stent) may be a sock-like structure so that the expandable element is inserted into the lumen of the expanded scaffold after the scaffold has been placed within the target site. In this configuration, the scaffold is not fixably attached to any component of the expandable element. The expandable element may be an angioplasty balloon catheter, an intravascular lithotripsy (IVL) balloon catheter, a drug coated or drug eluting balloon, or an expandable element with other function depending on the clinical situation and target lesion configuration. A preferred method may comprise a single procedure that utilizes different balloon catheters with different therapeutic effects sequentially within the same unattached scaffold. The method, hence, may include at least some the following operations. The device is inserted and deployed proximal to the target site to provide support for distal navigation to the target site. During the distal navigation, the self-expandable scaffold may expand against the vessel wall to provide support for distal navigation. Subsequently, the scaffold is recovered by the outer sheath and re-positioned at the target site where the expandable element is slowly and gradually expanded over a time period of at least 90 seconds, but preferably over 2-3 minutes to a pressure in the range of 5-6 atm. This action will expand the scaffold against the wall to the desired diameter. The expandable element may be an IVL balloon catheter and acoustic pulses may be delivered to the vessel wall. The expandable element is then collapsed to restore blood flow, leaving the scaffold to support the expanded vessel for a prolonged period of time greater than 5 minutes. In some embodiments, the expandable element can be removed from the scaffold after being collapsed. In some embodiments, the expandable element may be then exchanged for a second expandable element comprising a drug coated balloon. The second expandable element can be expanded for a prescribed amount of time to transfer an agent to the vessel wall and then collapsed (and may be removed) while the scaffold remains expanded. Subsequently, the scaffold is collapsed and the scaffold is removed.

By utilizing the balloon to expand the temporary stent (or the scaffold), not only the pressure of the balloon is brought to bear on the obstruction, but (i) its positive actions, e.g., the ability to expand the obstructive lesion gradually in a controlled manner with incremental increasing pressure, can be enhanced and (ii) its negative actions, e.g., the tendency of the balloon to expand uncontrollably in a sudden and abrupt manner creating injury and dissections and the tendency for the unfolding of the leaves of the balloon during expansion to cause intimal tears and dehiscence, both of which may predispose to restenosis and to vascular recoil, can be diminished by the overlying temporary stent structure and the method of utilizing such. The wires of the temporary stent or separate scoring members may provide areas of focal force on the plaque that will allow the plaque or obstruction to be dilated with less pressure creating a controlled expansion compared to the uncontrolled rupture and dissections frequently seen with POBA. The scaffold essentially protects the vessel from the balloon unfolding injury while preventing areas of focal rapid expansion which may damage the vessel wall. There may be a more gradual stretching and more gradual deforming of the smooth muscle cells, and they may have an opportunity to accommodate this stretching and maintain their integrity rather than being irreparably injured as is frequently the case with POBA. Further, with the scaffold placed over the expandable element, the unfolding of the leaflets of the expandable element as it expands may not cause intimal tears, dehiscence, or damage (as is frequently the case with POBA) as the scaffold protects the intima from contact with the expandable element. This is a critical aspect of the current method as intimal tears and damage are the precursors of dissections and the precursors of the reparative cellular proliferation process, both of which predispose the patient to restenosis. The instant method of combining a slow, incremental expansion over at least 90 seconds and the intimal injury protection offered by the removable scaffold from intimal injury from the unfolding of the expandable element along with applying a means of focal force and dilating with lesser pressures may cause less injury to the vessel wall, and, hence, less of a reparative neointimal hyperplastic response that results in restenosis.

Therefore, the expandable element, e.g., a balloon, can serve two distinct functions: 1) it can dilate the plaque or obstruction (and in a more consistent manner because of the overlying temporary stent structure), and 2) it can dilate the temporary stent more effectively, with more force, and with more lumen gain than could be achieved by dilating the temporary stent structure without the assistance of the balloon. The overlying temporary stent structure also protects the vessel from the damage of the expandable element, e.g., the balloon.

Therefore, utilizing the instant method together the expandable element, e.g., a balloon and the temporary stent structure may be able to effectively dilate the vessel safely and effectively and then support the dilated vessel subsequent to the dilatation for a time period adequate to promote plastic modulation and the reshaping of the tissues that results in a consistent and sustainable lumen gain not achievable with current methods of revascularization.

In some embodiments, the first elongate member comprises an outer, actuator sleeve and the second elongate member comprises an inner, balloon catheter shaft to which the balloon can be mounted. In some embodiments, the temporary stent comprises a porous braided stent structure. In some embodiments, the temporary stent structure comprises a laser cut stent.

Treating advanced vascular disease is one of the largest health care expenses born by society. Many simpler, less expensive interventional methods, such as POBA, are frequently not effective, necessitating the use of more complex and expensive alternatives, such as stenting and surgery, which cost billions of dollars each year.

The method of the present invention is expected to improve on the results of POBA and reduce or avoid the need for stenting and/or surgery, by causing less vascular injury initially, preventing elastic recoil that frequently demands stenting, and preventing restenosis by simultaneously administering a non-proliferative agent. Procedures conducted according to many embodiments of the present invention are expected to cost significantly less than POBA and the various devices and maneuvers designed to correct the complications of POBA.

An aspect of the present invention provides a medical device for treating a target site in a body lumen. Often, the target site will be a region of a blood vessel occluded with plaque. The medical device may comprise a stent-like structure which may comprise a plurality of scoring filaments or elements and a plurality of non-scoring filaments or elements. The scoring filaments and the non-scoring filaments may be interwoven with one another, but may not be. Alternatively or in combination, at least some of the scoring and non-scoring filaments may not be interwoven with one another. For example, the filaments may be struts of a laser-cut scaffold. The plurality of non-scoring filaments may be configured to radially support the body lumen after the stent-like structure has been expanded therein. The stent-like structure may comprise more non-scoring filaments than scoring filaments. By providing more non-scoring filaments or elements than scoring filaments or elements, greater structural strength and improved scoring by the stent-like structure can be provided. Scoring of a lesion or occlusion in the body lumen, for example, may depend on providing a focal force at one or several areas upon and within the plaque. If too many filaments are scoring filaments, this focal force would be diluted or divided over all of the filaments and none may be dominant in directing force into the plaque to cause the plaque to fracture beneath the scoring filaments. Thus, only a limited number of scoring filaments or elements may be needed to produce the desired results of a few areas of focal fracturing of plaque present in the body lumen such as a blood vessel. In some embodiments, the scoring filaments or elements can be substantially flat, and not projecting radially from the scaffold, when the stent-like structure is in a collapsed configuration. In some embodiments, the scoring filaments or elements may not score a vessel wall when the stent-like structure is being navigated to the target site. In some embodiments, the scoring filaments or elements can be transformed to be substantially perpendicular to a vessel wall when the stent-like structure is expanded thereby providing scoring to the plaque or lesion. In some embodiments, the stent-like structure may not comprise scoring elements.

The stent-like structure typically has an expanded configuration and a collapsed configuration. The stent-like structure in the expanded configuration may be collapsible into the collapsed configuration. The stent-like structure in the expandable configuration can be collapsed into the collapsed configuration by axially shortening the stent-like structure.

The medical device may further comprise an expandable element. The stent-like structure may be disposed over the expandable element, or the expandable element may be inserted into the lumen of the stent-like structure. The expandable element may be expandable to urge the stent-like structure radially outward against an inner wall of the body lumen. The expandable element may comprise an inflatable balloon. The expandable element may be independently collapsible from the stent-like structure. The expandable element may be or may not be fixably attached to the stent-like structure. The expandable element may comprise an outer surface coated with a therapeutic agent or substance. The expandable element may comprise one or more pores for releasing a therapeutic agent or substance.

The medical device may further comprise a catheter shaft, and the expandable element may be disposed over the catheter shaft.

Another aspect of the present invention provides methods of treating a target region (or site) in a body lumen. Often, the target site will be a region of a blood vessel occluded with plaque. A medical device, e.g., a catheter assembly disclosed herein, may be positioned at or near a target region in the body lumen. An expandable element of the catheter assembly may be expanded (i) to expand a stent-like structure (or scaffold) disposed over the expandable element and (ii) to urge both the expandable element and the stent-like structure against an inner wall of the target region. In some embodiments, the inner wall of the body lumen may also be scored with one or more of the stent-like structure or the expandable element. A therapeutic agent may be released from an outer surface of the expandable element for infusion into the inner wall. Alternatively or in combination, a therapeutic agent may be released from one or more of pores of the expandable element, from the stent-like structure (or scaffold), or the scoring elements of the stent-like structure (or scaffold) for infusion into the inner wall. In some cases, the expandable element may comprise an energy delivery means/device or energy generating device. The expandable element may be collapsed to leave the stent-like structure expanded against and radially supporting the inner wall. The stent-like structure may be left expanded against the inner wall for a period of time of at least 3 minutes to cause one or more of (i) inhibiting elastic recoil of the inner wall, (ii) allowing the therapeutic agent to infuse into the inner wall, (iii) inhibiting or minimizing flow limiting dissections, or (iv) to transmitting energy or motion to the inner wall. After a period of time, the stent-like structure may be collapsed over the collapsed expandable element and the entire medical device removed from the body lumen.

Alternatively, the expandable element may not be fixably attached to the stent-like structure so that different expandable elements with different therapeutic actions may be inserted alone or sequentially to provide one or more of the following: expansion of the vessel, intravascular lithotripsy, delivery of some other energy source, or drug/substance delivery.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1-6 illustrate exemplary devices and procedures in accordance with many embodiments of a prior art invention.

FIG. 1 is a side view of a stent-like temporary scaffold or structure according to many embodiments of a prior art invention.

FIG. 2 is a magnified top view of the interwoven filaments of a stent-like temporary scaffold or structure according to many embodiments of a prior art invention.

FIG. 3 is a magnified perspective view of non-scoring filaments interwoven and received within a notch of a scoring filament in a stent-like temporary scaffold or structure according to many embodiments of a prior art invention.

FIG. 4 is a side view of a scoring filament having a plurality of notches for receiving a plurality of non-scoring filaments in a stent-like temporary scaffold or structure according to many embodiments of a prior art invention.

FIGS. 5A-5C are side views of a plurality of scoring members of a scoring filament in a stent-like structure according to many embodiments of a prior art invention.

FIG. 6A is a cross-sectional view of an occluded blood vessel prior to treatment in accordance with many embodiments of a prior art invention.

FIG. 6B-6D are cross-sectional views of a stent-like temporary scaffold or structure disposed over an expandable element as used to treat the blood vessel occlusion of FIG. 6A in accordance with many embodiments of a prior art invention.

FIG. 6E is a cross-sectional view of the occluded blood vessel of FIG. 6A after the treatment of FIGS. 6B-6D in accordance with many embodiments of a prior art invention.

FIG. 7 shows perspectives of scoring and non-scoring elements.

FIGS. 8A and 8B are strain curves of current device and current device with energy means.

FIGS. 9A-9D represent the current invention and inserting energy means within it.

FIG. 10 demonstrates energy delivery device incorporated into a device.

FIG. 11 illustrates a method of utilizing the invention with an option of inserting and utilizing energy means to improve suboptimal results.

FIG. 12 illustrates the instant invention and connections to a controller which may be a programmable pump to inflate/deflate the balloon and/or rapidly change the pressures, a controller for energy delivery to the device or scaffold, or both.

FIGS. 13A-13D illustrate exemplary stent-like structures with attenuating or non-attenuating components.

### DETAILED DESCRIPTION OF THE INVENTION

The following description will typically be with reference to specific structural embodiments and methods. It is to be understood that there is no intention to limit the invention to the specifically disclosed embodiments and methods but that the invention may be practiced using other features, elements, methods and embodiments. Preferred embodiments are described to illustrate the present invention, not to limit its scope, which is defined by the claims. Those of ordinary skill in the art will recognize a variety of equivalent variations on the description that follows. Like elements in various embodiments are commonly referred to with like reference numerals.

The prior art, other than prior art by the current inventor, does not address a removable balloon stent apparatus that dilates the plaque and supports the wall after plaque dilatation. Lashinski et al. in U.S. Pat. No. 6,773,519 describe a stent like device which is deployed and then removed, and describes a removable coupler which is part of the device, but not a removable stent. Tsugita in U.S. Pat. No. 6,652,505 describes a guided filter which may be used to deliver a stent and removed, but not a removable stent. Kahmann in U.S. Pat. No. 5,879,380 describes a device and method for relining a section of blood vessel that has been injured or removed, not a device to both dilate the lesion and prevent elastic recoil as does the many embodiments of the present invention discussed below. Those patents and applications are incorporated herein by reference.

At least five separate but complementary actions of the current disclosure and method can be achieved relative to the inside surface of a blood vessel. The method, device, and system provided herein can provide a time proven balloon action to effectively dilate the occlusion. The method, device, and system provided herein provides a scaffold over the balloon to prevent the intimal tears (caused from the balloon unfolding) and prevent the abrupt expansion, both of which predispose to dissections and the resultant restenosis from the excessive reparative hyperplastic process. As disclosed herein, scoring members can more evenly apply stresses on the plaque and allow expansion at lesser pressures and thus cause less dissection and injury. As disclosed herein, the scaffold, acting independently of the balloon, acts as a transient, removable stent to lessen acute elastic recoil. The method, device, and system provided herein can inhibit restenosis, e.g., when combined with a drug delivery. Prior art devices and methods do not possess the mechanical advantages provided by many embodiments of the present invention, i.e., the scaffold with scoring members to create crevices that allow the plaque to be more homogeneously compressed at lower pressures with less injury while providing channels for an antiproliferative or other agent to perfuse into the media of the vessel, and the ability of the scaffold to be used as a transient, temporary, or removable stent to reduce the incidence of acute elastic recoil and flow limiting dissections, and acting in concert with the agent to prevent restenosis. Drug eluting and drug coated balloons may provide an advancement over plain old balloon angioplasty (POBA) by lessening the incidence of restenosis from 40-60% to 10-15%. However, restenosis from neointimal hyperplasia is only one of three major problems associated with POBA. The other two, acute elastic recoil and flow limiting dissections, are not addressed by drug eluting and drug coated balloons. Hence, in current practice, there is still a need to place a stent to address these two major problems when they occur, and they occur in up to 40-50% of cases in some studies. In most of these cases, “bail out” or unintended stenting will be needed to address the acute recoil and the flow limiting dissections that occur. The recoil and dissection phenomena are so common that many practitioners actually proceed directly to stenting to save time and complete the intervention with good results rather than attempt to perform just an angioplasty prior to determining the need for stenting. This can add cost to the procedure and leave a metal structure in the patient which may cause other problems such as late stent thrombosis, in-stent restenosis, stent fracture, stent malapposition, and others. These sequalae of the stent placement can often be very difficult to address when they occur. Hence the drug eluting and drug coated balloons are only a partial solution to the dilemma. Utilizing them with the embodiments according to the current invention and methods, however, can provide a complete solution to the three major deficiencies of POBA. Moreover, improvements may comprise the addition of adjunctive energy generating/delivery and transmitting means to enhance the mechanical actions by the device, the system and the method provided herein and weaken the ‘calcific matrix,’ as calcifications within the plaque or vessel wall will be referred to subsequently.

Aspects of the method and device disclosed herein can address all three primary deficiencies of POBA without the need for stenting or foreign materials to be permanently placed within the vessel to maintain a channel for blood flow. Restenosis is addressed, as are drug eluting and drug coated balloons, by providing delivery of a drug or other substance to inhibit restenosis, whether by a drug eluting balloon or a drug coated balloon, or with a drug coated onto the stent-like structure or the scoring elements of the stent-like structure. Many embodiments in accordance with the present invention support the wall of the vessel after dilatation to prevent or severely lessen the occurrence of flow limiting dissections. Many embodiments in accordance with the present method also support the wall while the drug is acting on the smooth muscle cells to cause them to relax and hence prevents acute elastic recoil. By performing these three actions, the need for stenting will be significantly lessened if not eliminated in most cases. This is not the case when utilizing drug eluting or drug coated balloons without the current invention. Additionally, adjunctive energy delivery means, not present in prior art versions of this invention, may be combined with the other features to destabilize the structure of the calcific matrix.

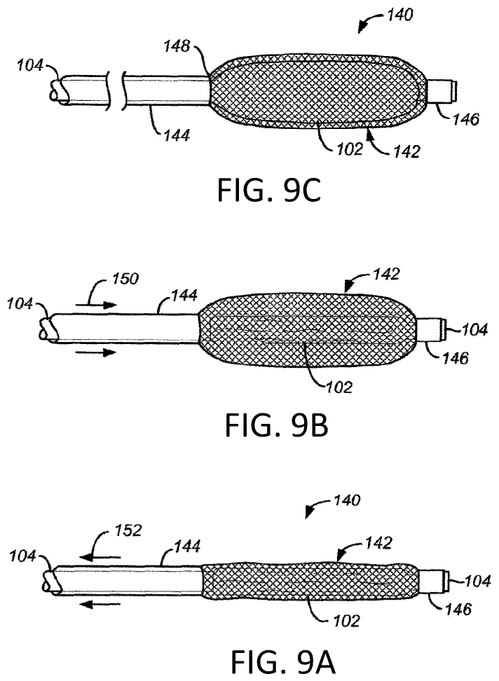

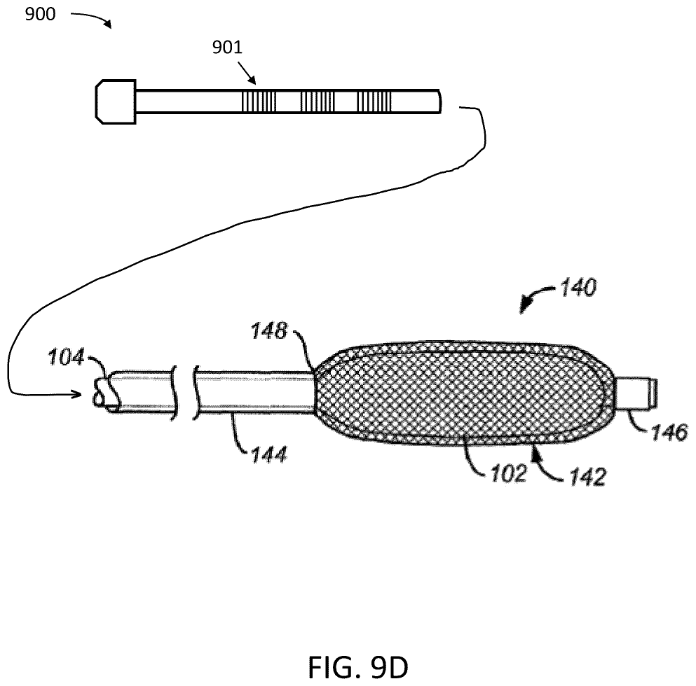

For reference, FIG. 9C demonstrates the dual shafts of the device and the expandable balloon element fully expanded with the scaffold overlying it. Balloon stent assembly 140 includes balloon catheter shaft 104 with balloon 102 at its distal end and an actuator sleeve 144 surrounding the balloon catheter shaft 104. A radially expandable temporary stent (e.g., a braid) 142 can be positioned over the balloon 102. The balloon 102 and the temporary stent 142 are shown expanded in FIG. 9C. The distal end 146 of the temporary stent 142 may be secured to the distal end of the balloon catheter shaft 104 while the proximal end 148 of the temporary stent 142 may be secured to the distal end of actuator sleeve 144. The temporary stent 142 can be removed from the patient following the procedure.

FIG. 9B demonstrates the balloon collapsed and the scaffold remaining expanded so that it provides distension to the vessel wall. By moving the actuator sleeve 144 distally in the direction of arrows 150, the temporary stent 142 may become expanded over the collapsed balloon 102, as shown in FIG. 9B, and can stay expanded when the balloon 102 is deflated and collapsed. The temporary stent 142 may be fixed to the catheter shaft 104 distally, but not to the balloon 102. It is in this expanded state of temporary stent 142 and collapsed state of balloon 102 that the temporary stent can act as a stent like structure and allow blood flow to be restored.

FIG. 9A demonstrates the scaffold to be collapsed over the collapsed balloon element for insertion and removal from the target site. By moving the actuator sleeve 144 proximally in the direction of arrows 152, the braid 142 may be contracted against the deflated and collapsed balloon 102, and may even help lower the profile of the collapsed balloon. It is in this contracted state that balloon assembly 140 is typically inserted and removed. The scaffold is normally expanded with the balloon, but when the balloon is contracted or collapsed to allow for distal blood flow to recommence, the scaffold can be locked into an expanded configuration by manipulating catheter shaft 104 and actuator sleeve 144 with one's fingers. The instant method of leaving the scaffold expanded for several minutes while blood flow is restored distally may cause the smooth muscle to accommodate the stretch of the angioplasty. This may diminish the incidence of acute elastic recoil, one of the major acute problems of POBA. In fact, prolonged expansion of the vessel has this desired effect; however, the time that a balloon can be left expanded is limited as ischemia may develop because the balloon occludes blood flow to distal tissues. The current method will support the expanded vessel with the stent-like structure and provide for distal blood flow to obviate ischemia developing during the expansion phase.

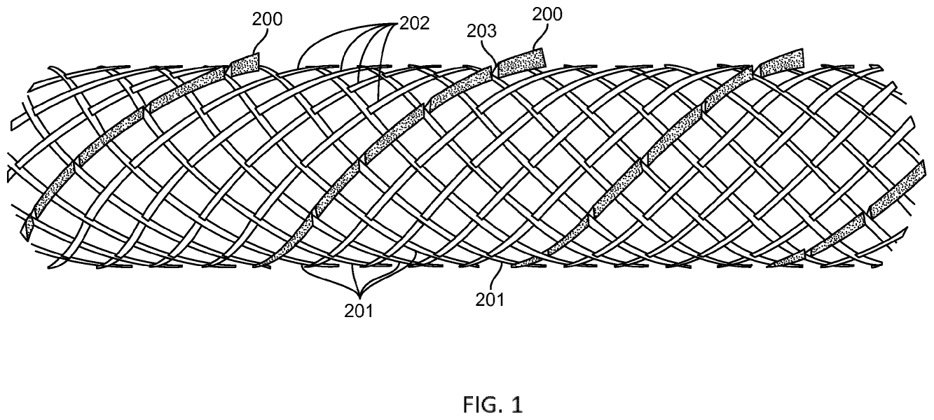

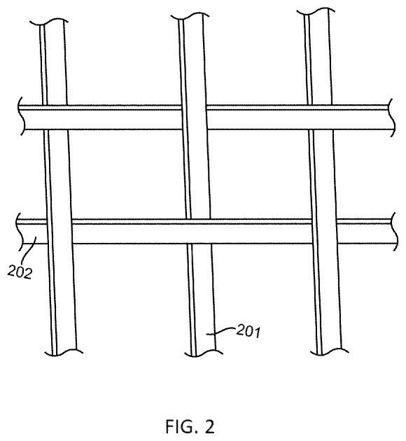

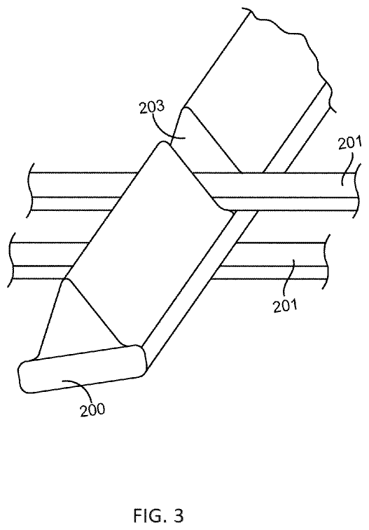

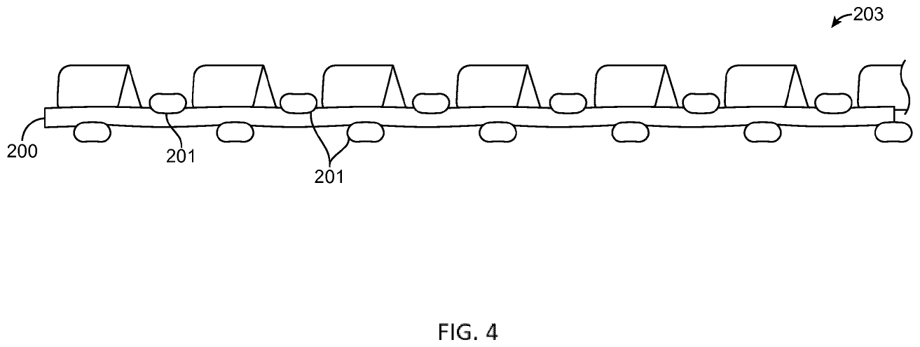

FIG. 1 shows an exemplary scaffold of a prior art device comprising scoring wires 200 and supporting wires 201, 202. FIG. 2 shows expanded view of a section of the braid device containing only the supporting wires 201, 202. To conserve space, the cutting or scoring wires or members 200 may measure 0.005″ to 0.009″ in greatest radial dimension or height to the peak of the triangle, all oriented in the same direction (scoring members not crossing other scoring members) and the supportive non-scoring members 201, 202 measure only 0.001″ to 0.005″ in greatest radial dimension or height. This may cause the thicker cutting or scoring wires or members to overlap only the thinner supportive wires or members. FIG. 3 demonstrates an expanded section of FIG. 1 in which two rather flat supporting members 201 are interwoven with a scoring member 200. The flat supporting members 201 are present within the notches 203 mentioned above. FIG. 4 demonstrates a section taken along the course of one scoring braid member 200. One can appreciate that the scoring member 200 may cross over, and may be crossed by, the other supporting members 201. FIG. 4 shows that instead of the abrupt notches, there may be rounded indentations or other configurations (not shown) within the scoring members 200 to accommodate the crossing members 201. The series of notches create a serrated configuration of relatively pointed ends of the scoring members between the notches. The serrations formed by the series of notches will score the lesion/vessel more easily and more completely than a round or rectangular scoring member.

The number of filament members of the braid will affect the radial strength as well as the pics per inch (the number of wire intersections per inch), the size of the central mandril, the rate of advancement of the mandril as well as other factors. Hence, any number of combinations of the type of wire or filament, shape and dimension of the wire or filament, relative stiffness of the wire or filament, the number of the types of wires or filaments, the total number of wires or filaments, the total number of the structurally important wires or filaments, mandril size, the braid density, the pics per inch, the direction of the thick and thinner members, and other factors are possible and will contribute to the size, strength and functionality of the scaffold. By utilizing these variables, one can optimize the cutting or scoring features, the strength of the device, and limit the size, all important goals.

In the case of both non-braided and braided scaffolds, including a laser cut scaffold, some of the struts of the scaffold may be constructed so that while most all of struts are more or less configured in a cylindrical orientation when the device is contracted or collapsed for insertion, that upon expansion some part of the struts, including the apex, or some of the struts may be oriented more or less perpendicular to the vessel wall. These more or less perpendicular struts, or members of the stent, will act as a scoring element when compressed into the plaque by the expansion of the balloon against the stent. When contracted or compressed for removal, these struts or parts of the struts may return to the more or less cylindrical shape. Other struts or parts of struts may be consistently oriented in a conventional cylindrical pattern to provide strength to the scaffold.

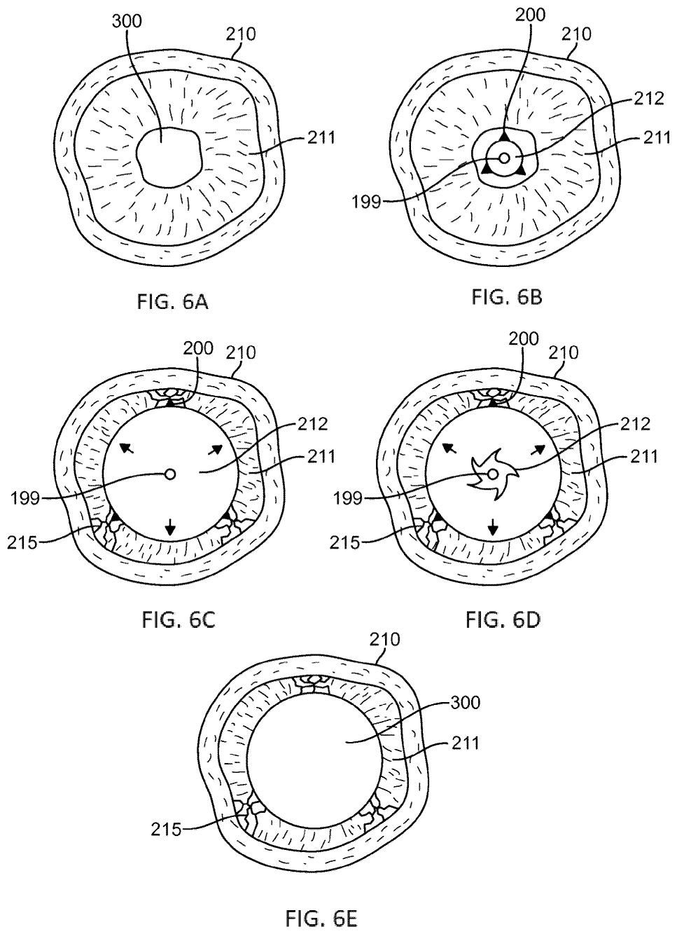

FIGS. 6A-6E show cross sections of a blood vessel 210 with partially occluding plaque 211 and a vessel lumen 300.

Paclitaxel acts on the cytoskeleton or microtubules within smooth muscle cells by enhancing polymerization and causes the smooth muscle cells to relax. There are other cellular effects, certainly, but the dysfunctional microtubules are thought to be reason the smooth muscle cell relaxes rather than contracts as a result to exposure to certain drugs. Sirolimus acts on the potassium channel mechanism to similarly inhibit contraction of the smooth muscle cells. Existing methods do not maintain the blood vessel in an expanded state for a period of time sufficient for the drug or substance to be administered by a component of the device and delivered to the vessel wall, the drug or substance to transit through the vessel wall to the media, and to act upon the smooth muscle cells to inhibit contraction. Most methods of drug delivery expand the lesion rather quickly (less than 90 seconds) with a POBA balloon and then leave the balloon expanded for another 90 seconds or so and then collapse and remove that balloon. A drug coated balloon then may be inserted and expanded for a short period of time to deliver the drug from the surface of the balloon to the intima (typically one minute or less) and then removed. Hence, there is no time available for both the vessel to remain in the desired expanded state while the drug transits the vessel wall to the media and acts upon the smooth muscle cells to prevent smooth muscle cells from contracting and recoiling to a smaller lumen diameter. The administered drug may inhibit the neointimal hyperplasia that contributes to restenosis but does little to prevent or inhibit acute vascular recoil. In some embodiments, employing a temporary stent may prolong the distension of the blood vessel after the drug delivery (for at least 3 minutes but preferably for 8-12 minutes, and possibly for several hours or days) by the temporary scaffold 142 (FIGS. 9A-9C). In some embodiments, the temporary stent combined with paclitaxel or sirolimus or other drugs/substances may provide enough time of prolonged distension of the vessel for the drug or substance to act upon the cytoskeleton and microtubules so that the smooth muscle cells may not contract upon the removal of the temporary stent. Many embodiments and methods of the present invention may take advantage of paclitaxel or other antiproliferative drug or substance through the use of the scaffold 142 (or other stent structure) acting as a temporary stent to provide this action of prolonged expansion, allowing the drug or substance to act upon the cells so that they may not contract when the temporary stent is removed. Without the prolonged expansion, the drug may not have enough time to act upon the cellular components to cause the smooth muscle cell to relax. The extra time provided by the expanded temporary stent while blood is flowing through the area along with the uptake and action of the drug may result in diminished elastic recoil of the vessel, and better long term patency.

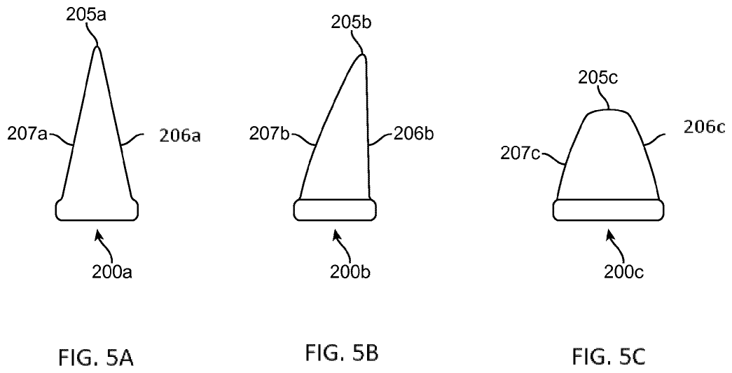

Each scoring filament may have one or more of a pointed, triangular, or rectangular shape. FIGS. 5A-5C show exemplary shapes of a plurality of scoring members of a scoring filament in a stent-like structure.

As disclosed in the present disclosure, means may be present which cause the scoring tip of the scoring members to be oriented substantially perpendicular relative to the longitudinal orientation of the scaffold when expanded but oriented more or less parallel to the longitudinal axis of the scaffold when the scaffold is collapsed. These means may comprise a shape at the base of the scoring element or within the notches which, upon the expansion of the expandable balloon element, causes the orientation of the scoring element to change from a non-scoring orientation more or less parallel to the scaffold surface and longitudinal axis of the scaffold to a scoring orientation more or less perpendicular to the scaffold surface and the longitudinal axis of the scaffold. This may be accomplished by constructing the base of the scoring element with a rounded edge or with additional scallops extending into the base so that pressure may cause the scoring element to rotate nearly 90 degrees from its resting position. The non-scoring element may also have scalloped edges that may allow the base of the scoring element to rotate from a perpendicular orientation with respect to the surface of the scaffold to a relatively flat position when there is no pressure upon the scaffold members when the scaffold is contracted or collapsed. The notches of the scoring elements may also be configured with a waist or thinner cross sectional dimension than the base which would facilitate rotation of the scoring member in relation to the non-scoring member. Simply, with no outward radial force on the scaffold, the scoring members are configured so they are oriented with their long axis and apex lying flat in more or less the same plane as the non-scoring filaments or members. Upon inflation of the expandable element and resultant pressure placed upon the non-scoring members which compress the base of the scoring members from opposing sides, the scoring members respond to the compression by rotating so they are oriented more or less perpendicular to the long axis of the scaffold and the non-scoring members with the apex of the scoring member directed away from the scaffold.

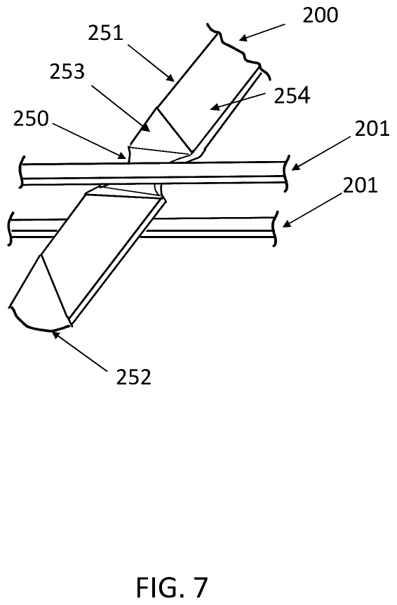

As demonstrated in FIG. 7, each scoring filament or member 200 may have a base part 252 configured to cause the orientation of the scoring members to rotate when the expandable element is inflated to expand the scaffold and compresses the base part of the scoring member. The scoring filament 200 may comprise a tip or apex 251 and two substantially flat surfaces e.g., 254 (the opposite surface not shown). When the expandable element is not inflated or expanded, the scoring filament 200 may be oriented so the surface 254 is substantially parallel to the surface of the scaffold, thus the scoring tip 251 is not pointing to a vessel wall. This may eliminate any scoring or contact of the scoring member on the vessel wall. The scoring member 200 may be configured with indentations or scalloped cut outs 250 that allow rotation or bending of the scoring members 200 in relation to the supporting members 201 (e.g., non-scoring members). When the scaffold or stent is contracted and there is no outward pressure on the stent by the expandable member, the scoring filaments or members assume a position with the long axis of each scoring member is substantially in the same plane as the supporting or non-scoring members of the stent-like structure. In FIG. 7, the scoring member 200 may be configured with curved or scalloped shapes or waists e.g., 250 in the notch area 253 which are configured to receive the non-scoring filament e.g., 201 when no pressure of the expandable element upon the non-scoring or supportive members is provided. This may serve two purposes: 1) it may diminish the overall profile of the device when not expanded as the scoring elements, e.g., the apex 251 of the scoring filament 200 may not be directed outward, and 2) it may minimize the potential for damage to the vessel from the inadvertent exposure of scoring elements when navigating or positioning the device. Other configurations (not shown) are also contemplated, and the examples herein are representative of the possibilities.

As shown in FIG. 7, the notch 253 in the base of the scoring member 200 may be configured so that when the non-scoring filaments of the scaffold are under tension in an expanded or expanding scaffold position, the scoring member may be compressed by the non-scoring members 201. In some cases, the non-scoring members may be interwoven under and over the scoring member. In some cases, the non-scoring members may not be interwoven under and over the scoring member. The base 252 of the scoring member 200 may be asymmetrically rounded in a manner so that compression by the expandable element on the asymmetrically rounded base may cause the scoring member to rotate from a low profile orientation with the scoring element long axis relatively flat to a greater profile orientation with the scoring element projecting away from the scaffold into a scoring position. Hence, the means that promotes change of orientation of the scoring members from low profile to a perpendicular scoring position may comprise one or more of: the base of the scoring element, the notch within the scoring element, the non-scoring element, heat setting of any element, the outward radial pressure from the expandable element, expansion of the inner dimension of the scaffold, or translation of the inner and outer shafts which puts the scaffold into compression. In the latter case, compressing the braided scaffold may cause the crossing angles to change as the filaments or members of the braid expand radially. Means within the non-scoring members, the base of the scoring filaments, or the notch of the scoring filaments may be present to change the orientation of the scoring filaments when the scaffold is compressed or shortened, irrespective of pressure upon it.

Shallow notches within the non-scoring members 250 may also be present to receive the scoring member and facilitate the change in orientation of the scoring members.

Alternatively, the non-scoring filaments may not cross over and under the scoring filaments and may not be received by the notches nor reside within the notches. In other words, the non-scoring filaments or elements may not cross, nor be received within, the notches of the scoring filaments in other configurations. In this alternative embodiment, the scoring and non-scoring filaments may not be inter-related. In this alternative configuration, which may be present in the case of the scaffold being a laser cut or non-braided stent-like structure, the base of the scoring element may be configured similar to FIG. 7. In this case, the pressure of the expanding element may cause the scoring elements to change orientation. In some cases, the instant temporary stent may not comprise scoring elements at all.

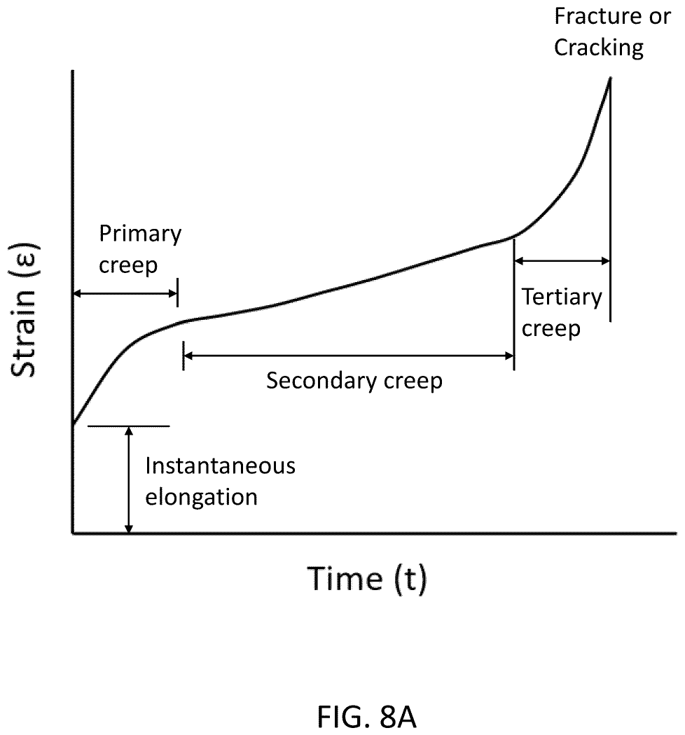

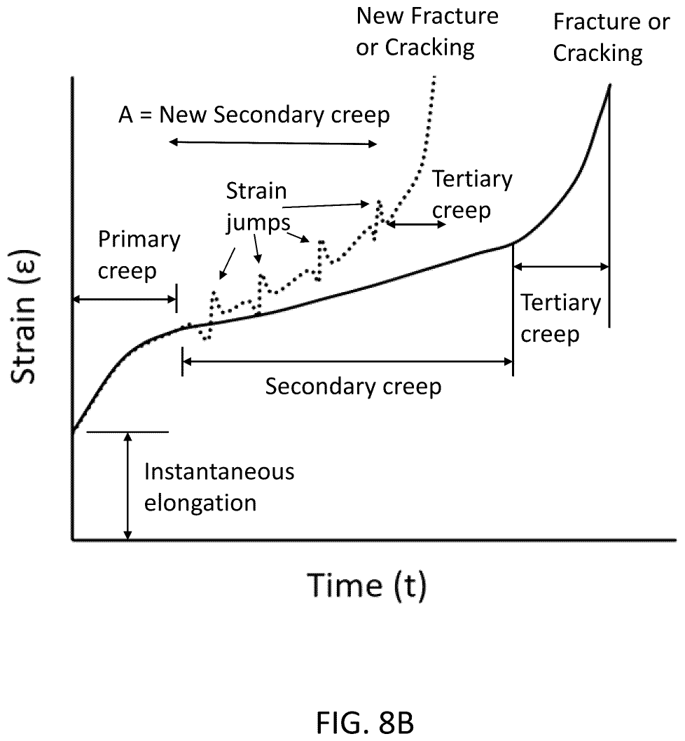

Improvements to the prior art will result in the device of the present invention to comprise novel means and methods of slowly expanding the expandable element and temporary stent-like structure with one or more of adjunctive methods including pressure alterations, vascular lithotripsy, laser, or other energy transfer to the vessel wall while the expansion is gradually occurring or has occurred. This differs from current methods which expand the lesion before administering energy to the vessel wall. Simultaneous energy delivery to the vessel wall while the expandable element and scaffold with or without scoring means are expanding will be more effective in disrupting and rearranging the calcified plaque and medial calcifications for more lumen gain. Further, the method of the delivery of pulses of energy to the vessel wall after the temporary scaffold has supported the vessel wall for several minutes will enhance the creep phenomenon which is dependent on applying a strain over a period of time. Simply, the addition of an adjunctive energy means to deposit energy as “strain jumps” in the calcified plaque and vessel wall will accelerate the tertiary creep and fracture/cracking of the calcified material and cause the calcified area to fracture more quickly and easier than a constant force alone. This phenomenon is known as strain jumps and creates structural instability within the material subject to the strain resulting in fracture with a shorter duration of strain. This is shown in FIGS. 8A and 8B. FIG. 8A demonstrates the accumulation of strain over time and the creep phenomenon that occurs until the substance finally develops loss of structural integrity causing it to fail. The substance may be the calcified matrix in the instant case. In FIG. 8B, the adjunctive energy deposition may be either intermittent or at specific times during the dilatation cycle or during the extended supportive cycle when the balloon is normally collapsed. In the latter case, the balloon may be expanded if needed to deliver the energy source. Instead of a constant force supplied by either the expandable element or by the scaffold with scoring elements for a period of time as in the prior art, the improvements in these embodiments will comprise additional means and methods of energy delivery so that there is a constant force but augmented by additional forces administered either intermittently or at specific intervals. In FIG. 8B, the projected strain curve with the addition of the strain jumps is superimposed upon strain curve of FIG. 8A and demonstrates the addition of adjunctive energy, or strain jumps, the new secondary creep phase (A), and a new tertiary creep. The time to fracture or cracking representing compressive instability in the calcific matrix is significantly shortened after the addition of strain jumps from the administration of adjunctive energy.

The adjunctive energy may be comprised of one or more of several forms of energy. One form is to utilize energy propagation means within the solution that expands the balloon expandable element. A method of utilizing a removable balloon expandable scaffold to treat a target site using microbubbles, substances, dissolved gases, or nanoparticles as cavitation nuclei within the solution used to inflate the balloon and administering sonic or other energy to this solution to create cavitation and subsequent pressure waves that are transmitted to the vessel wall where they weakens the integrity of the calcific matrix by cavitation and resultant pressure waves in combination with one or more of other means of inducing instability in the calcific matrix including the scoring elements, creating strain jumps with adjunctive energy delivery or alteration, longitudinal motion of catheter or scaffold, prolonged strain upon the vessel wall with prolonged distension, or physical motion is claimed. Rapid pulses of laser energy may also be utilized to deliver energy to the media and create structural instability in the calcific matrix.

Recent advances in intravascular lithotripsy (IVL) are welcome improvements to vascular intervention. However, the method has several deficiencies and usually demands pre-dilation angioplasty of the lesion prior to the IVL placement of the device and activation of the IVL device, and subsequent post IVL angioplasty and/or drug coated balloon (DCB) angioplasty and stenting. And, with IVL, there is no means to combat vascular recoil despite multiple steps in the procedure. The balloon leaves unfold against the arterial intima and still may produce intimal dehiscence and tears which have the potential to evolve into dissections or represent a nidus for initiation of the neointimal hyperplastic restenosis cascade. Hence, improvements are needed to correct the deficiencies of IVL and the multistep process by which plaque modification occurs.

With one embodiment, the improved device of the current invention, the advantages of a temporary scaffold (no intimal dehiscence, no/limited recoil, time for plastic deformation to occur, less injury to the vessel, etc.) are integrated with the advantages of IVL (disruption of calcified material for better compression of plaque and calcified vessel wall and the resultant improved compliance).

Finally, the stent-like structure may be collapsed after the period of time and the complete catheter assembly including the collapsed balloon and collapsed stent-like structure may then be removed from the target region.

FIG. 8A illustrates the effect of applying a constant strain to a somewhat rigid object (such as the calcified matrix). This is the expected result of the protracted distension of the vessel by the removable scaffold. It is likely that fracture of cracking of the calcified matrix will occur in most cases.

FIG. 8B illustrates the effect of applying a constant strain to a somewhat rigid object (e.g., calcific matrix) plus adding adjunctive energy delivery, including motion, to the system. The fracture or cracking will occur earlier with the adjunctive energy as the added adjunctive energy will enhance and accelerate the strain effects of the expandable element and scaffold. Hence, the addition of adjunctive energy means, in one form or another, will be beneficial to the process of compressing even heavily calcified vessels. Both atherectomy, with its potential for distal embolization, and the multistep intravascular lithotripsy process, which demands a series of actions consisting of pre-dilation maneuvers which may injury the vessel, energy deposition, dilation after energy deposition, and separate application of a drug coated balloon, may be avoided since all of the necessary actions for lumen gain will be incorporated into the device of the current invention.

The sum of these advantages, i.e., the mechanical advantages of slowly expanding the balloon, of the braid and scoring elements in dilating the plaque with less pressure, less dissection, and less injury along with the temporary stent providing prolonged distension to overcome vascular recoil combined with integral drug elution to inhibit restenosis combined with energy deposition to enhance the other actions will significantly improve patient outcomes.

Moreover, the temporary stent may be used with other modalities other than energy or drugs, such as stem cell implants and exosomes, plant substances, minerals, radiofrequency, lithotripsy, electroporation, heat, atherectomy, gene therapy, cryotherapy, electrical pulses and currents, radiation, iontophoresis, other pharmacological agents and substances, and the like. The concept is to employ multiple modalities which directly or indirectly affect or weaken the calcific matrix and other components of the plaque and/or vessel wall which may inhibit compression of these structures and the resultant expected lumen gain. Other modalities comprised within the method or device may affect other components of the plaque or vessel wall to inhibit intimal tears, dissections, injury to the vessel, vascular recoil, vascular compliance, and restenosis. By incorporating multiple different efficacious modes of action specifically directed to different components of the problem within one device, more complete and more efficacious results can be obtained. The compliance of the vessel may be maintained after a treatment, something that does not occur with stenting or with incomplete compression of the calcific matrix. Utilizing multiple different devices can be avoided, which will improve the procedure as to the safety profile, time for the procedure, risk to the patient, and cost to the health care system.

The prior art device comprised scoring, temporary stenting and prolonged distension, and simultaneous drug delivery. The current improvements comprise a means and method of gradually and incrementally inflating the expandable member to gradually expand the lumen in a novel manner, novel means and methods of delivering adjunctive energy means to weaken the calcific matrix, novel methods and maneuvers of delivering motion, stress, and strain to the target site to weaken the calcific matrix, and utilizing the scaffold to delivery energy or substances to either weaken the calcific matrix or inhibit recoil and restenosis or both. Novel means of depositing energy to the vessel wall may also be used with the temporary stent structure to weaken the calcified matrix and other structures in the plaque and vessel wall. These means augment the pressure by the balloon and/or the temporary stent structure including one or more of the following: acoustic generators, including ultrasonic and sub-sonic generators, intravascular lithotripsy, electrical generators, radial pressure wave generators, and vibratory, electrohydraulic, iontophoretic, reversible or irreversible electroporation, electromagnetic, piezoelectric, compressive, and concussive means amongst others. Combining energy depositing means with the inherent advantages of the current device may further enhance the effectiveness and overcome the problems and downsides of isolated energy applications. For example, intravascular lithotripsy (IVL) utilizes a semi-compliant balloon to distend the plaque/vessel. This process subjects the intima to intimal dehiscence from the shear forces caused by the unfolding of the balloon during expansion which frequently form the nidus for dissections to occur. There is also no gradual balloon expansion typically and no means for prolonged vessel distension to obviate vascular recoil. Scoring is not provided with IVL Drug delivery must be provided by a standalone drug coated balloon with a separate application after the IVL dilates the lesion rather than being incorporated into the dilating device. Hence, standalone IVL may be successful in weakening the calcific matrix, but it causes problems that it does not address and is an incomplete solution.

The method of utilizing the current invention after IVL or atherectomy to fully compress the already weakened or partially removed calcific matrix, respectively, and achieve maximal lumen gain is also claimed. This would entail inserting the current invention after IVL or atherectomy to the target site, expanding the expandable element and scaffold for a period of time, collapsing the expandable element, and leaving the scaffold expanded to support the vessel wall for a second period of time, and collapsing the scaffold, and removing the entire device. A drug may be delivered during the expanding the expandable element step, during the time the scaffold is expanded, or both. Vascular recoil will be inhibited by either the prolonged distention of the vessel, the drug delivery, or both. Vascular restenosis will be inhibited by the drug, energy delivered, the lack of injury to the vessel, the prolonged distension or a combination of two or more of these features.

A preferred embodiment of the present disclosure may comprise an energy delivery device incorporated into the expandable element. The energy delivery device may comprise a plurality of energy generating means. The energy delivery device may be placed within the cavity of the expandable element and may be affixed to the shaft of the expandable element. The method may comprise advancing the device to the target site, gradually and incrementally expanding the expandable element to contact the vessel wall and expand the scaffold and the vessel wall. Adjunctive energy may then be delivered to the vessel wall at select times before, during, or after the expanding step. The expandable element may be an integral part of the device, i.e., fixably attached to the scaffold, or may be a separate device that is configured to be inserted into the partially expanded scaffold. The energy delivery device may be intravascular lithotripsy.

A second preferred device configuration and method of the present disclosure may comprise separate expandable elements and scaffolds that are not fixably attached to one another. The expandable element may be configured to be inserted into the lumen of the at least partially expanded scaffold, which may be self-expanding. The method may comprise advancing the scaffold to the target site, at least partially expanding a self-expanding scaffold at the target site, inserting an expandable element which may comprise a balloon and the energy delivery device into the lumen of the at least partially expanded scaffold, expanding the expandable element, delivering energy to the vessel wall before, during, and/or after the expanding step, collapsing the expandable element while leaving the scaffold expanded against the wall for a period of time of at least 3 minutes, preferably 8-12 minutes and potentially several hours to days, and collapsing the scaffold. The expandable element with the energy delivery device may be removed at any time after expansion and energy delivery in this method.

To incorporate energy generating means (or energy delivery device/means) into other configurations of the device disclosed in the present disclosure, one configuration may comprise a separate energy generating means that is inserted into the inner lumen of the expandable element and positioned for optimal energy delivery as an add-on device. The method of doing so may entail placing the device at a target site, at least partially inflating the expandable element, inserting the energy delivery device into the lumen of the expandable element before or after partial expansion, delivering a form of energy from the energy delivery device to the vessel wall, collapsing the expandable element, leaving the scaffold expanded for a period of time of at least 3 minutes, collapsing the scaffold, and removing the entire device from the patient.

The methods may comprise delivering energy at specific times during any stage, but particularly the latter stages, of the gradual expansion of the expandable element and/or upon full expansion of the expandable element. Hence, in some cases, the energy depositing means may not be an integral part of the device but may be inserted into the device and activated during and after the gradual expansion of the device. This is demonstrated in FIGS. 9A-9D. As in FIG. 9D, the energy delivery device 900 comprising energy generating means 901 may be sized to fit within the inner lumen of the expandable element, and positioned at an appropriate location prior to energy delivery. A conductive medium may be inserted between the shaft of the energy generating means and the inner surface of the inner shaft of the catheter. This location inside of an outer catheter would necessitate that the energy delivering means transmits energy or discharges sufficient to pass through the shafts of the overlying catheter, the shaft comprised of a material that does allow or promote the transmission of energy. Frequently, however, the catheter material necessary for the catheter to function properly may not readily transmit the energy. In this case, through “windows” or plugs of energy conductive material (such as structural hydrogel, polyurethane, other polymers, metals, conductive materials, and the like) may be inserted into specific locations within the catheter wall which allows generated energy to propagate through these energy transmitting windows in the overlying reconstituted catheter shaft and into the fluid filled expanded balloon where one of vaporization, cavitation, shock wave type forces, or sonic pressure waves are generated and transmitted into the vessel wall whereby the energy wave will augment the mechanical expansion actions of the expandable element or the calcium disruptive actions of the scoring elements of the temporary scaffold or both. By combining both of those means, i.e., mechanical scoring and mechanical pressure waves into one device and into one method, more complete and homogeneous plaque and calcific matrix compression will occur resulting easier and more complete compression by the expandable member and in better lumen gain at less pressures and with less vessel injury in doing so. In the case of the acoustic or energy transmissible material windows that are placed within the two catheter shafts of the current invention, a segment of the catheter wall may be removed and replaced with a different material, or a different material may be placed at a specific site upon extrusion of the shaft initially. These windows (not shown) may be circumferential around the catheter shaft or may only represent “cutouts” that occupy ⅓ or less of the circumference. They may be of any desired shape and size that allows for transmission of energy through the windows without weakening the integrity of the catheter shaft. The length of the transmissible windows may be only 1 cm or so and sized to fit over just one energy generating/delivery component or may be sized to fit over all of the energy generating/delivery components. In the case of focal acoustic windows within the catheter wall, they may be positioned over each energy generating/delivery component. In the current invention, since there are two shafts of the catheter, the windows or acoustic cutouts will be placed in each inner and outer shaft so that they are aligned when the two shafts are positioned for sub maximum or maximum expansion of the expandable element. Water, saline, or some other energy conductive material may be placed or injected between the multiple catheter shafts to facilitate energy conductivity. If the catheter can be constructed of materials which transmits the desired energy, the windows or special means to ensure transmission may not be needed.

A method of treating a vascular channel may comprise inserting an energy delivery device into the inner lumen of a temporary stent device comprising a scaffold and an expandable element, positioning the energy delivery device adjacent to acoustic windows within the catheter shafts capable of transmitting energy, at least partially expanding the expandable element and scaffold, activating the energy delivery device for a specified period of time and delivering energy to the vessel wall, fully expanding the expandable element, collapsing the expandable element, leaving the scaffold expanded for at least 3 minutes, and collapsing the scaffold.

The alteration of transmitted energy by the scaffold or stent-like structure can be problematic in some cases. Stents themselves may not directly attenuate shockwaves in lithotripsy procedures. Instead, the presence of a stent in the blood vessel may have secondary effects on the propagation of shockwaves due to interactions with surrounding tissues and fluids.

When intravascular lithotripsy shockwaves encounter a stent within a blood vessel, several factors come into play:

Reflection and Scattering: The presence of a stent can cause reflection and scattering of shockwaves. Depending on the material and design of the stent, some of the shockwave energy may be reflected back towards the source or scattered in various directions. This can affect the distribution and intensity of shockwaves reaching the target area, potentially altering treatment efficacy.

Acoustic Impedance Mismatch: Stents may create acoustic impedance mismatches at the interfaces between the stent and surrounding tissues or fluids. These mismatches can lead to partial reflection of shockwaves and changes in wave propagation patterns, influencing the distribution and attenuation of shockwave energy.

Interference with Targeting: In some cases, the presence of a stent may make it more challenging to accurately target and deliver shockwaves to the intended treatment site. Stents may obstruct the path of shockwaves or cause shadowing effects, potentially affecting treatment outcomes.

Overall, while stents themselves may not directly attenuate shockwaves in lithotripsy procedures, their presence can impact shockwave propagation through secondary effects such as reflection, scattering, and acoustic impedance mismatches. Understanding these interactions is important to optimize lithotripsy treatments in patients with stents. The composition of the stent or scaffold is critical to either prevent any alteration of shock waves, whether to enhance or inhibit the effect of these shock waves due to their physical properties and interactions with acoustic energy. The scaffold or stent may be comprised of different materials to inhibit or attenuate shock waves. The scaffold or stent may comprise metals such as stainless steel, titanium, and nitinol that may be used in stents, clips, and prosthetic implants. These metals can attenuate shockwaves through mechanisms such as reflection, absorption, and scattering. The degree of attenuation depends on factors such as the type of metal, its thickness, and its acoustic properties. The scaffold or stent may comprise polymers, including various plastics and synthetic materials. Polymers can attenuate shockwaves through absorption and scattering mechanisms. The composition, density, and thickness of the polymer material influence its ability to attenuate shockwaves. The scaffold or stent may comprise ceramics such as alumina (aluminum oxide) and zirconia due to their biocompatibility and mechanical properties. Ceramics can attenuate shockwaves through absorption, scattering, and reflection. The composition and microstructure of the ceramic material affect its acoustic properties and attenuation characteristics. The scaffold or stent may comprise composite materials, which consist of combinations of different substances such as fibers, resins, and fillers, that can be engineered to have specific acoustic properties. Composite materials used in medical devices or implants may attenuate shockwaves through a combination of absorption, scattering, and reflection mechanisms. The scaffold or stent may comprise hydrogels. Hydrogels are water-absorbent polymer materials that may be used in various medical applications, including tissue engineering and drug delivery. Hydrogels can attenuate shockwaves through absorption and scattering mechanisms, as the water content and structure of the hydrogel influence its acoustic properties.

Overall, the attenuation of shockwaves by materials in lithotripsy procedures depends on factors such as the composition, density, thickness, and acoustic properties of the materials involved, as well as the size of the cells or spaces between the struts or structural components of the stent. Closed-cell stents are characterized by small free cell areas between the struts and the cells are frequently surrounded by the struts, whereas open-cell stents have larger uncovered gaps and the cells are open areas may not be completely surrounded by the struts.

No matter the location of the energy delivery device (or energy generating means/device), whether incorporated into the scaffold device, as a separate component inserted into the lumen of the at least partially expanded scaffold, or as a separate component that is inserted into the lumen of the shaft of the expandable member, the energy generated and transmitted towards the vessel wall may be significantly attenuated or shielded by the scaffold which is placed between the expandable element and the vessel wall. Hence, the effective energy delivered to the vessel wall and the calcific matrix may be diminished by the overlying scaffold in many cases, or the energy delivery may not be uniform or in a desired pattern. To overcome this deficiency of placing a scaffold over the energy delivering expandable element, one or more of scaffold configurations may comprise: 1) an open cell stent pattern that minimizes the structural components of the scaffold and maximizes the open areas which cause no or less attenuation, 2) a scaffold of a material which transmits the energy without significant attenuation of the energy, 3) windows or apertures in the scaffold for the energy to be transmitted therethrough without attenuation, and 4) a pattern of non-attenuating windows within the scaffold oriented to provide maximum energy transmission therethrough and with purposeful attenuating means in other areas of the scaffold so that the energy may be applied to the vessel wall in a desired pattern that comprises both maximum and minimum energy delivery areas in the vessel wall adjacent to the windows and the attenuating components, respectively. While the attenuating means may not attenuate the energy completely, the relative difference between the areas of maximum transmission and minimum transmission may be significant. Similar to the focal force description previously regarding scoring, applying an energy source focally or in a focal pattern to the vessel wall and the calcific matrix is more effective than applying a generalized force or energy source that may be rather homogeneous to the entire vessel wall and to the rather cylindrical calcific matrix which may cause the calcified matrix to expand more or less concentrically to absorb the energy pulse and only develop structural instability or fracture of the calcific matrix at much more intense, frequent, or repetitive energy depositions. Hence, the windows or apertures created in the scaffold of the present disclosure may be configured to deliver the energy in a pattern that maximizes the delivered energy to one or more areas of the vessel wall and minimizes the energy delivery to other areas of the vessel wall. This disparity and difference in the energy delivery with the devices and methods of use to different sections of the wall and matrix may be more effective at causing structural instability in the plaque and calcific matrix than delivering a homogeneous energy force to the vessel wall. Hence, with the device and technique disclosed herein, the pulse intensities may be diminished (which is potentially safer), fewer pulses may be needed (which saves time and costs), and a pattern which causes a predictable fracture pattern in the calcific matrix that allows the vessel to expand more easily and predictably than with random energy delivery. While there may be areas in most scaffolds devoid of any attenuating material (between the braid filaments or between the stent struts), a closed cell configuration of the scaffold may limit the transmission of energy. The predictable fracture pattern may create fragments of the calcific matrix which more easily are expandable and then orient with the other fragments more easily. Simply, the random fracture pattern with the IVL (and potentially other energy delivery devices) creates a shock wave that randomly cracks the calcific matrix leaving some large fragments intact and resistance to expansion without further injuring the vessel. The scaffold of the present disclosure is designed to deposit energy maximally in patterns that promote more or less uniform sized fragments that allow the vessel to expand more easily and to maintain this expansion after the temporary scaffold is removed.

In some cases, it may be beneficial to simply limit the size and configuration of the struts to minimize the shadowing, reflection, scattering, impedance mismatch, targeting interference, and/or attenuation of transmissible energy and to maximize the open space devoid of any substance to enhance the transmission of energy therethrough.

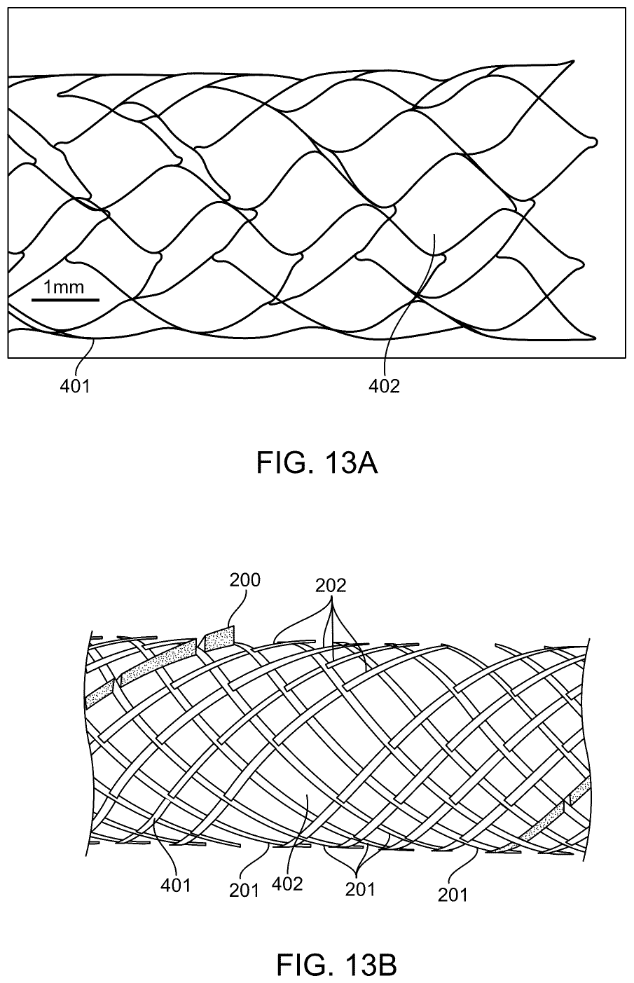

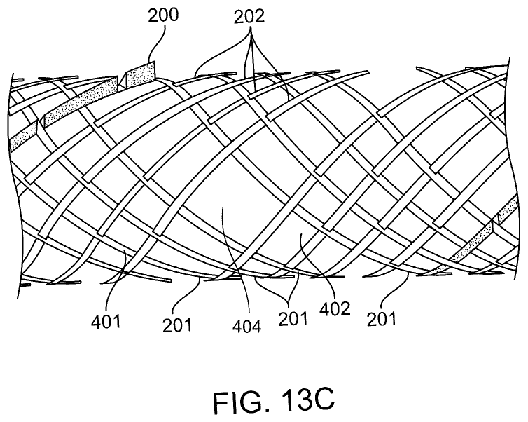

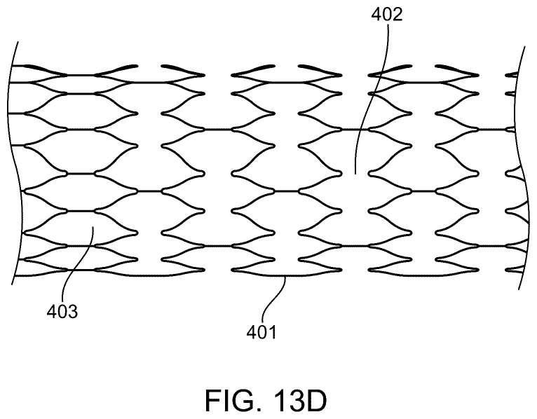

Examples of the patterns of attenuating and non-attenuating components of the scaffold are detailed in FIGS. 13A-13D. In some embodiments, the struts of the stent may be flat to cover more space to attenuate energy or they may be thicker to purposefully minimize energy delivery in certain areas.

FIG. 13A demonstrates a stent structure with thin struts 401 that may not significantly inhibit the transmission of energy and large open areas 402 which may allow the energy to be transmitted without inhibition of energy transmission. This may be a preferred embodiment when utilizing the current invention with various forms of energy delivery devices. FIG. 13A shows a closed cell configuration comprising braid filaments e.g., 401 and open area 402. In some embodiments, the stents can be in an open cell configuration.

In some embodiments, the scaffold may purposefully comprise attenuating means that may limit the energy transmission to certain areas of the vascular wall while allowing full transmission of energy to the vessel wall through open apertures designed to fracture the calcific matrix. The disparity in energy deposition caused by this configuration may cause the calcific matrix to more easily fracture and to fracture in a more or less predictable pattern rather than subjecting the entire vessel to a uniform energy deposition in which there is no control over where or how the calcific matrix may fracture. This configuration may provide focal areas of more intense energy force deposition adjacent to areas of diminished energy force deposition that may limit the intensity, duration, or number of energy pulses needed to fracture the calcific matrix into fragments that allow the calcific matrix and the vessel to be more expandable and compressible. The following descriptions are only examples of potential configurations designed to deliver energy in a pattern or arrangement which fractures the calcific plaque in a more clinically beneficial manner than otherwise. FIG. 13B demonstrates a tubular braided stent comprised of one of wide and/or dense braid members 201, 401, and 202, a density of pics per inch that inhibit the transmission of energy, and open areas 402 created by omitting opposing members in one spiral braid direction. FIG. 13B shows a closed cell configuration, with scoring members 200, but with enlarged cells created by omitting one braid filament 202. This creates a spiral arrangement of larger open windows 402 for uninhibited energy transmission therethrough. Supporting members 201 and 202 may be configured with a wider width (not shown) and other properties which inhibit energy transmission, thereby providing a spiral pattern of maximal energy deposition while inhibiting energy transmission and deposition in the remainder of the vessel beneath the scaffold structure. FIG. 13C demonstrates a tubular braided stent comprised of one of wide and/or dense braid members 201 and 202 with a density of pics per inch that inhibit the transmission of energy, and open areas 402 and 404 created by omitting opposing members in both braid spiral directions. FIG. 13D demonstrates an open-cell laser cut type stent comprising sections or areas of energy inhibiting scaffold components or struts combined with areas of cutouts or absence of scaffold components or struts which transmit energy freely to the vessel wall in a desired pattern providing areas of minimal energy transmission and areas of maximum energy transmission to the vessel wall, respectively.

In some embodiments, the open areas (e.g., 402) can be created by increasing the distance between adjacent supporting members or filaments. In some embodiments, the open areas can be created by removing a supporting member or filament. In some cases, the supporting members or filaments may comprise a material which vibrates upon energy transmission, the vibratory energy transmitted to the vessel wall to further cause structural instability and other effects in the calcific matrix and other components of the vascular wall.

One configuration in which the scaffold is optimized to deliver a pattern of energy pulses when the scaffold is tubular braid from a 16 carrier braider comprises removing two opposing filaments each from each of eight right and eight left rotating filaments. This would produce an intersecting spiral configuration of area of maximum energy deposition. Utilizing a closed cell or open cell laser cut stent or a mesh braid as the scaffold would produce small diamonds of maximum energy delivery. In some cases, a closed cell stent which minimizes the free cell area may adequately deliver a desirable pattern. In some cases, a scaffold with areas of full energy transmission and areas of attenuated to absent energy transmission may be more effective at dilating the calcific matrix subsequent to the energy deposition. The smaller areas of energy deposition through the open cells between struts of the scaffold may augment larger areas of energy deposition through a patterns of windows created in the scaffold.

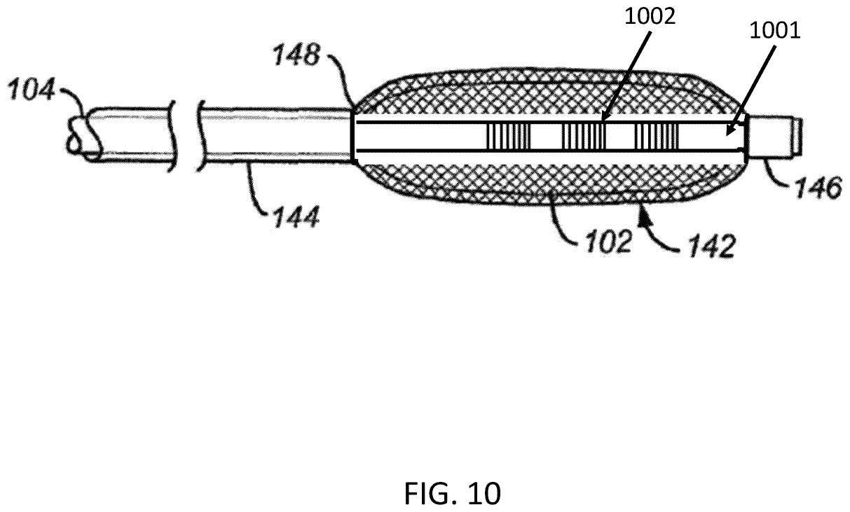

In some cases, the energy delivery device (or energy generating means/device) may be physically incorporated into and a part of the device disclosed herein. In this embodiment, the energy delivery device may be an integral part of the expandable element and may be incorporated into the device so that the energy generating means is placed onto or within the shaft immediately adjacent to and surrounded by the interior of the expandable element. The energy delivery means may be placed immediately adjacent to the cavity of the expandable element in this instance as shown in FIG. 10. FIG. 10 illustrates the removable scaffold with the energy delivery device incorporated into the expandable element. When the energy delivery device is incorporated into the device, it may not be visible from the outside. For illustration purpose, in FIG. 10, a portion of the scaffold is removed to show the incorporated energy delivery device. The energy delivery device 1001 comprising energy generating means e.g., 1002 can be inserted to the expandable element 102 after the expandable element 102 is expanded and the temporary stent or scaffold 142 is expanded by the expandable element. The energy delivery device 1001 can be inserted through the inner lumen of the shaft 104. 144 shows the actuator sleeve of the shaft 104. 148 shows the proximal end of the temporary stent or scaffold 142.

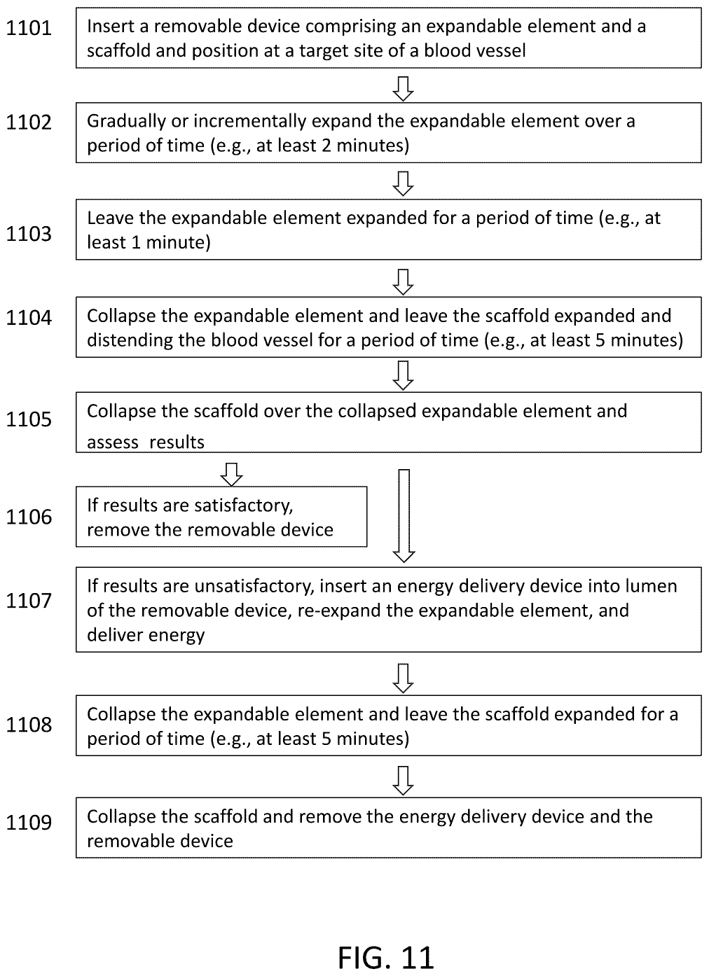

Further, the method of treating a target site or lesion with the device comprising an expandable element and a temporary stent (or scaffold) without any adjunctive energy delivery device initially, assessing the results, and then retreating the lesion by inserting the energy delivery means into inner catheter lumen, expanding the expandable element and scaffold, and treating while either the expandable element or the scaffold is expanded or both are expanded is also disclosed. This method would reserve the expensive energy delivery component for those cases with potential suboptimal results and this method would essentially be a “bail out” procedure rather than a primary intervention. Hence, savings to the healthcare system may be achieved while providing better patient care. This schematic is illustrated in FIG. 11. FIG. 11 demonstrates a method of treating a target site. The method can comprise a process of 1101, e.g., inserting a removable device comprising an expandable element and a scaffold and positioning the device at the target site of a blood vessel. The method can comprise a process of 1102: gradually or incrementally expanding the expandable element over a period of time. In some embodiments, the period of time can be at least about 2 min. The method can comprise a process of 1103: leaving the expanded element expanded for a period of time, e.g., at least about 1 min. The method can comprise a process of 1104: collapsing the expandable element and leaving the scaffold expanded which may distend the blood vessel for a period of time, e.g., at least about 5 min. The method can comprise a process of 1105: collapsing the scaffold over the collapsed expandable element and assessing treating results. If the results are satisfactory, the method can comprise a process of 1106: removing the removable device. If the results are unsatisfactory, the method can comprise a process of 1107: inserting an energy delivery device into lumen of the removable device, re-expanding the expanding element, and delivering energy from the energy delivery device. The method can comprise a process of 1108: collapsing the expandable element and leaving the scaffold expanded for a period of time, e.g., at least about 5 min. The method can comprise a process of 1109: collapsing the scaffold and removing the energy delivery device and the removable device.

The method in this instance may entail positioning the energy delivery means over the conductive windows in the catheter shafts as discussed above. In some cases, the method may comprise a sock-like stent-like structure that is separate and is not fixably attached to the expandable element. In some cases, the energy delivery may be accomplished during or after the expansion of the expandable element.

In some embodiments, the present disclosure provides a method of diminishing recoil and restenosis at a target site in a blood vessel, comprising: (a) advancing a medical device to the target site, wherein the medical device comprises (i) a stent-like structure comprising a plurality of scoring members and a plurality of non-scoring members, wherein the plurality of non-scoring members is more than the plurality of scoring members, (ii) an expandable element capable of expanding the stent-like structure against a wall of the body vessel, wherein the plurality of scoring members is configured to score and penetrate into a plaque and/or a wall of the blood vessel with focal forces, (iii) an inner and an outer shafts, wherein a distal end of the stent-like structure is attached to a distal end of the inner shaft and a proximal end of the stent-like structure is attached to the outer shaft, wherein the expandable element is disposed in and operatively coupled to the inner shaft, wherein translation of the inner and outer shafts collapses the stent-like structure over a collapsed expandable element for removal from the body vessel; (b) gradually inflating the expandable element to press the stent-like structure against the wall of the blood vessel and expand the blood vessel; (c) applying an adjunctive energy source; (d) collapsing the expandable element to provide flow of blood distally; (e) leaving the stent-like structure expanded supporting the vessel wall for greater than 3 minutes; (f) collapsing the stent-like structure; and (g) removing the expandable element and the stent-like structure.

In some embodiments, the present disclosure provides a method of diminishing recoil and restenosis at a target site in a blood vessel, by: (a) providing a self-expanding stent-like structure or scaffold comprising a plurality of scoring members and a plurality of non-scoring members, wherein the stent-like structure comprises more non-scoring members than scoring members, said scaffold attached only to a proximal delivery shaft member and, when deployed in expanded configuration, provides a lumen for an expandable member to be inserted and positioned within the lumen of the scaffold, the expandable element capable of expanding the stent-like structure against the wall of the body lumen, wherein the scoring members are configured to score and penetrate into the plaque and/or vessel wall with focal forces, (b) inflating the expandable element to press the stent-like structure against the vessel wall and expand the body lumen, (c) applying an adjunctive energy capable of causing structural instability in the calcific matrix or applying a drug/substance source or both, (d) collapsing the expandable element to provide flow of blood distally, (e) leaving the stent-like structure expanded supporting the vessel wall for greater than 3 minutes, (f) collapsing the stent-like structure, and (g) removing both the expandable element and stent-like structure from the blood vessel.

In some embodiments, the inner shaft may comprise an energy delivery device. In some embodiments, the energy delivery device may be mounted on the inner shaft adjacent to or within a cavity of the expandable element. In some embodiments, the energy delivery device may not be enclosed in the inner shaft. In some embodiments, the energy delivery device may be a standalone device that can be inserted in the inner shaft during the treatment. In some embodiments, the energy delivery device may be inserted coaxially within an inner lumen of the inner shaft of the medical device.

In some embodiments, the inner and outer shaft walls may comprise focal areas of material inserts capable of transmitting sufficient energy from the inner lumen of the inner shaft to the cavity of the expandable element to create one or more of (1) vaporizing the fluid within the expandable element, and (2) generating sonic pressure waves which are further transmitted through the soft tissues and act upon the calcific component to fracture the calcific components.

In some embodiments, the stent-like structure may comprise material or materials capable of inhibiting energy transmission from the energy delivery device. In some embodiments, the energy delivery device is intravascular lithotripsy.

In some embodiments, a method disclosed herein can comprise applying a drug or substance from a drug coated expandable element or the stent-like structure or the scoring elements of the stent-like structure.

In some embodiments, the method can further comprise removing the expandable element while leaving the stent-like structure expanded. In some embodiments, the method can further comprise inserting a second expandable element comprising a different therapeutic modality and expanding the second expandable element within the lumen of the scaffold for a sufficient period to deliver the therapy. In some embodiments, the method can further comprise collapsing the second expandable element. In some embodiments, the second expandable element comprises either a drug delivery balloon or a balloon capable of delivering energy sources to the vessel wall.

In some embodiments, the stent-like structure comprises scoring members. In some embodiments, the stent-like structure comprises non-scoring members or supporting members.

In some embodiments, the stent-like structure does not comprise scoring members.

In some embodiments, inflating the expandable element is gradual and incremental. In some embodiments, the inflating may take at least 90 seconds to inflate the expandable element to the target size or a pressure of at least 5 atm. In some embodiments, the gradual inflation is accomplished by a pump capable of incremental increasing the inflation pressure to the desired level and to alter the pressures abruptly thereby transmitting strain jumps to the vessel wall and components. In some embodiments, the pump may be controlled by a controller.

In some embodiments, the scaffold comprises acoustic windows devoid of any attenuating material to transmit acoustic pressure changes to the vessel wall. In some embodiments, the stent-like structure comprises a material capable of inhibiting transmission of acoustic pulses to the vessel wall by causing one or more of reflection, scattering, acoustic impedance mismatch, shadowing, and interference with targeting of the acoustic pulses. In some embodiments, the material comprises one or more of metals, polymers, ceramics, composite materials, and hydrogels. In some embodiments, the acoustic windows are configured in shapes and sizes to deliver maximum energy deposition to the vessel wall therethrough. In some embodiments, a relative placement of the acoustic windows and the inhibiting components of the stent-like structure are configured to provide areas of maximum energy transmission adjacent to areas of minimum or lessened energy transmission that create focal areas of energy deposition adjacent to focal areas of energy sparing to create structural instability within tissues affected by the acoustic pulses.

In some embodiments, the stent-like structure comprises configurations of: 1) an open cell stent pattern that minimizes the structural attenuating components of the stent-like structure and maximizes the non-attenuating open areas, 2) a scaffold of a material which transmits the energy without significant attenuation of the energy, 3) a scaffold comprising windows or apertures for the energy to be transmitted therethrough without attenuation, and 4) a pattern of non-attenuating windows within the stent-like structure oriented to provide maximum energy transmission therethrough and with purposeful attenuating means in other areas of the stent-like structure. In some embodiments, the energy is applied to the vessel wall in a desired pattern that comprises both maximum and minimum energy delivery areas in the vessel wall adjacent to the windows and the attenuating components.

Additionally, all methods of utilizing the configuration of the stent-like structure that is self-expanding and not attached to the expandable element may comprise an additional step of deploying the stent-like structure at a site in the blood vessel proximal to the target lesion to provide stability and an anchoring function to support distal navigation of guide wires and other devices through the target lesion and the distal vascular tree. In some cases, the target lesion is stenotic or completely occluded which may cause difficulty in traversing the target lesion with guide wires or therapeutic devices thereby creating difficulty with lesion access. The stabilization created by expanding the stent-like structure temporarily against the arterial/vessel wall proximal to the target lesion of the segment to be navigated may allow for enhanced pushability and support to the passage of wires and devices through tortuous or narrowed vessels. In some cases, this additional method may be employed only to cross the target lesion or stenosis with guide wires by temporarily deploying the stent-like structure proximal to the intended target, crossing the lesion with a guide wire, collapsing the self expanding stent-like structure, advancing the stent-like structure to the target site, and deploying the stent-like structure at the target site to dilate the stenosis according to at least one of the methods listed previously. Alternatively, after performing the supporting step in the above method, other therapeutic devices may be inserted through the inner lumen of the stent-like structure while the stent-like structure is deployed in an anchoring configuration. This may include POBA, atherectomy, laser treatment, plaque modification, IVL, or other modalities. After the therapeutic treatment is administered to the vessel, the therapeutic device may be removed. The stent-like structure may be collapsed and repositioned in the target lesion for further treatment. The expandable element may be inserted to expand the stent-like structure as in previously described methods. The expandable element may be collapsed and the stent-like structure left expanded against the vessel wall for a period of time of at least 5 minutes. Adjunctive energy or agents may be administered at some point. When utilizing this additional step, a stent-like structure that comprises either no scoring elements or scoring elements which do not rotate into scoring position may be desirable as scoring of the vessel wall proximal to the target site may be deleterious and therefore avoidable when feasible.

Further, an alternative method of utilizing the device for embolic protection may comprise positioning a stent-like structure and expandable element proximal to a lesion, expanding the expandable element so that it occludes blood flow, performing a therapy at the target site, aspirating the liberating debris from the therapeutic procedure, and collapsing the expandable element to restore blood flow.

Further, additional means and methods of diminishing the integrity of the calcific matrix and causing it to be more compressible for lumen gain and more permeable to delivered drugs or substances may be employed. While the energy delivery means described herein may be effective, the technology is expensive and may require specialized non-disposable equipment to support the energy generation and delivery. Other methods and means may be simpler and cheaper and include maneuvers that create longitudinal or rotational micro-movements of one or more of the device, expandable element, guide wire, or scaffold. This method may be performed by manual manipulation or by a mechanism (not shown) that is attached to the catheter shaft or guide wire. The persistent strain from prolonged distension will be accentuated by these micro-movements, as will the scoring actions, to create microfractures in the calcific matrix by utilizing this method. These methods may be utilized with other methods including energy delivery.

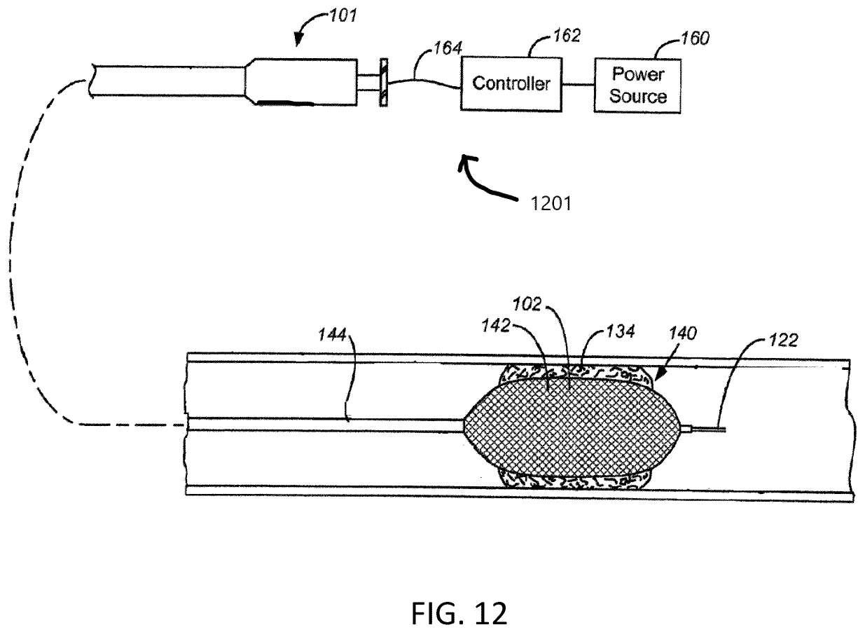

FIG. 12 demonstrates a programable controller module 1201 comprising a pump 101 that is programmed to inflate/deflate the expandable element at specified pressures and times. This controller may also comprise means for delivering the added energy source to the vessel wall or at least coordinated with a separate energy delivery system. The controller may comprise only means for delivery of energy in some cases. The inflation of the expandable element may be manual. The programable controller module 1201 can comprise an external power source 160 and a computer-based controller 162 by wires 164. The pump may inflate the expandable element 102 and the stent-like structure 142 of the device 140 can be expanded to contact a plaque. The device may comprise a guide wire 122 and a shaft 104. The pump can deliver pressure to the expandable element through the shaft 104.

The controller module may comprise a programmed balloon inflation pump that gradually and automatically expands the balloon to at least 4 atm in a series of steps comprising gradually and incrementally increasing the overall pressure in the balloon over at least 90 seconds, but preferably over two to three minutes. The programmable pump may inflate the inflatable element with either incremental repetitive pressure pulses or a continuous pressure increase over a period of time that is at least 90 seconds and preferably at least 2 minutes to achieve a target pressure within the expandable element of 6-7 atmospheres (atm). The method of expanding the lesion may comprise connecting a select configuration of the current device to the programmable controller module, programming the controller to inflate the inflatable element with either continuous inflation or intermittent pressure pulses to a pressure of no more than 7 atm over at least 90 seconds, inflating expandable element within the scaffold, collapsing the expandable element while leaving the scaffold expanded for a period of time of at least 3 minutes, but preferably 8-12 minutes, and collapsing the scaffold. When utilizing a program of pulsed intermittent increase in pressures, periods of quiescence with no increase in general overall pressure or pressure pulses may be present between the pulses.

Further, the controller module may be programmed to provide intermittent variable rapid pulses of different pressures including the absence of positive pressures. The altered pressure dips and peaks, a form of strain jumps, will cause a radial micro-motion within the gradually compressing tissues to augment the focal force scoring component and/or energy delivery by subjecting the calcium matrix to suddenly diminished force immediately followed by a rebound and subsequent increase in force over the former baseline.

With the gradually increasing pressures plus pressure pulses and the action of the scaffold on the plaque, providing focal force scoring, and giving the normal tissues a chance to accommodate the stretching and plastic deformation of tissues/plaque over a relatively protracted length of time of at least several minutes, the lesion/vessel will dilate with lower pressures, less injury. and less recoil.

Such a programable balloon inflation pump is a vital part of the system of the present disclosure. The pump is configured to be connected to a Leur like catheter fitting and to pump fluid into a catheter shaft and expand a balloon, and optionally to withdraw the fluid from the balloon. It is programmed to gradually and incrementally inflate an angioplasty type balloon (which in the instant case, will expand the overlying scaffold) over a period of 1-5 minutes, but typically 2-3 minutes. The gradual pressures changes may be linear or non-linear, and continuous or non-continuous, and may have interspersed episodes of sudden drop and restoration of pressures designed to create strain jumps as in FIG. 8B that lessen the integrity of the calcified matrix and allow more complete and homogeneous compression of the plaque. The controller module may also be programmed to provide longitudinal or rotational micro-movements of the device or scaffold, energy delivery, electrical pulses, and the like to create strain jumps which accentuate the mechanical effects of the expandable element.

The method of using a pump to inflate an expandable member and expand a temporary stent gradually to the point of full expansion with intermittent changes in pressure of greater than 0.1 atm is also claimed. The motion caused by the sudden changes in pressure will be transmitted to the calcific matrix and alter/weaken it by the scoring members and scaffold. Hence this novel method of inflating the expandable device with the insertion of strain jumps is designed to weaken structures within the plaque or vessel wall which resist compression by conventional methods of balloon expansion and prevent or inhibit optimal lumen gain.

In some cases, manual longitudinal micro-motion of the catheter shaft may cause subtle micro-longitudinal motion of the expanded scaffold. This will further serve to disrupt the calcium matrix and provide for more complete lumen gain. The method of providing longitudinal micro-motion may comprise placing the current invention at the target site, inflating the expandable element which expands the overlying scaffold, and one or more of providing manual longitudinal micro-movements of the catheter during inflation, at completion of inflation with expandable element and scaffold fully expanded, or after the expandable element has been collapsed leaving the scaffold expanded against the vessel wall. In some cases, some automated method of providing longitudinal or rotational motion to the catheter and/or scaffold may be provided.

In some cases, the controller module may comprise a generator element that provides an electric type current to the scaffold which acts upon and prevents at least a portion the smooth muscle cells from contracting by causing apoptosis or controlled cellular injury. Applying this current to the lesion may be a part of other methods.

The balloon stent assembly according to many embodiments of the present invention, in contrast with known temporary stents, can both dilate the plaque in a controlled manner using the balloon, which causes little injury to the vessel, and support the vessel for an extended period of time using the temporary stent in addition to providing adjunctive energy deposition means. Known temporary stents are commonly intended to only support the vessel after something untoward happens during the procedure, i.e., recoil, dissection, vasospasm, or vasoconstriction. Some also are designed primarily to facilitate the delivery of drugs to the arterial media. Many embodiments of the present invention, because all of the functions (protection against intimal injury, gradual dilatation and prolonged support functions, scoring, energy or mechanical force delivery, and drug delivery) happen more or less simultaneously, prevents dissections, vasospasm, vasoconstriction, and/or recoil as the vessel wall is supported during and immediately after the intervention, a great improvement over the prior art device. There is often virtually no time for the untoward events to occur with the current invention as there is no time that the vessel wall does not have radial force being exerted upon it. Hence, having multiple efficacious modes of action employed at virtually the same time is simpler, less risky, and less expensive with better results vs. the typical multistep procedural strategy of treating with POBA, then a plaque modification device, then a drug coated balloon or a drug coated stent. With the expandable element collapsed and the stent-like structure expanded, the vessel may maintain a distended configuration while blood flows through the stent-like structure and to the distal tissues, precluding ischemia that may develop if the vessel were occluded.

The method of treating a target site in a vascular channel with a single device comprising an expandable element, a removable scaffold that provides prolonged distension of the vessel for greater than 3 minutes, scoring elements, and adjunctive energy delivery means capable of weakening the integrity of the calcified matrix, and removing the device is claimed.

In some cases, the scoring element may not be present in the device. The above descriptions may have used terms above, below, top, bottom, over, under, et cetera. These terms may be used in the description and claims to aid understanding of the invention and not used in a limiting sense.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of diminishing recoil and restenosis at a target site in a blood vessel, the method comprising:
(a) advancing a medical device to the target site, wherein the medical device comprises:
(i) a stent-like structure comprising a plurality of scoring members and a plurality of non-scoring members, wherein a number of the plurality of non-scoring members is greater than a number of the plurality of scoring members,
(ii) an expandable element capable of expanding the stent-like structure against a wall of the body vessel, wherein the plurality of scoring members are configured to score and penetrate into a plaque and/or a wall of the blood vessel with focal forces,
(iii) an inner shaft and an outer shaft, wherein a distal end of the stent-like structure is coupled to a distal end of the inner shaft and a proximal end of the stent-like structure is coupled to the outer shaft, wherein the expandable element is disposed in and operatively coupled to the inner shaft,
wherein translation of the inner and outer shafts collapses the stent-like structure over a collapsed expandable element for removal from the body vessel;
(b) gradually inflating the expandable element to press the stent-like structure against the wall of the blood vessel and expand the blood vessel;
(c) applying an adjunctive energy source;
(d) collapsing the expandable element to provide flow of blood distally;
(e) leaving the stent-like structure expanded supporting the vessel wall for greater than 3 minutes;
(f) collapsing the stent-like structure; and
(g) removing the expandable element and the stent-like structure.

2. The method of claim 1, wherein the inner shaft comprises an energy delivery device.

3. The method of claim 2, wherein the energy delivery device is mounted on the inner shaft adjacent to or within a cavity of the expandable element.

4. The method of claim 2, wherein the energy delivery device is inserted coaxially within an inner lumen of the inner shaft of the medical device.

5. The method of claim 1, wherein the inner and outer shaft walls comprise focal areas of material inserts capable of:
(a) transmitting sufficient energy from the inner lumen of the inner shaft to the cavity of the expandable element to create one or more of vaporizing the fluid within the expandable element, and
(b) generating sonic pressure waves which are further transmitted through the soft tissues and act upon the calcific component to fracture the calcific components.

6. The method of claim 1, wherein the stent-like structure comprises material or materials capable of inhibiting energy transmission from the energy delivery device.

7. A method of diminishing recoil and restenosis at a target site in a blood vessel, the method comprising:
providing a self-expanding stent-like structure or scaffold comprising a plurality of scoring members and a plurality of non-scoring members, wherein the stent-like structure comprises more non-scoring members than scoring members, said scaffold attached only to a proximal delivery shaft member and, when deployed in an expanded configuration, provides a lumen for an expandable member to be inserted and positioned within the lumen of the scaffold,
inflating the expandable element to press the stent-like structure against the vessel wall and expand the body lumen, wherein the plurality of scoring members are configured to score and penetrate into the plaque and/or vessel wall with focal forces;
applying one or more of an adjunctive energy capable of causing structural instability in the calcific matrix or applying a drug/substance source;
collapsing the expandable element to provide flow of blood distally;
leaving the stent-like structure expanded and supporting the vessel wall for greater than 3 minutes,
collapsing the stent-like structure; and
removing both the expandable element and stent-like structure from the blood vessel.

8. The method of claim 7, wherein the expandable element comprises an energy delivery device.

9. The method of claim 7, wherein the energy delivery device comprises intravascular lithotripsy.

10. The method of claim 7, further comprising applying a drug or substance from a drug coated expandable element, the stent-like structure, or the scoring elements of the stent-like structure.

11. The method of claim 7, further comprising inserting a second expandable element comprising a different therapeutic modality and expanding the second expandable element within the lumen of the scaffold for a sufficient period to deliver the therapy.

12. The method of claim 11, further comprising collapsing the second expandable element.

13. The method of claim 11, wherein the second expandable element comprises either a drug delivery balloon or a balloon capable of delivering energy sources to the vessel wall.

14. The method of claim 7, wherein the stent-like structure comprises no scoring members.

15. The methods of claim 7, wherein the inflating of the expandable element is gradual and incremental and takes at least 90 seconds to inflate to the target size or a pressure of 5 atm.

16. The method of claim 15, wherein the gradual inflation is accomplished by a pump capable of incremental increasing the inflation pressure to the desired level and to alter the pressures abruptly thereby transmitting strain jumps to the vessel wall and components.

17. The method of claim 7, wherein the scaffold comprises acoustic windows devoid of any attenuating material to transmit acoustic pressure changes to the vessel wall.

18. The method of claim 7, wherein the stent-like structure comprises a material capable of inhibiting transmission of acoustic pulses to the vessel wall by causing one or more of reflection, scattering, acoustic impedance mismatch, shadowing, and interference with targeting of the acoustic pulses.

19. The method of claim of claim 18, wherein the material comprises one or more of metals, polymers, ceramics, composite materials, and hydrogels.

20. The method of claim 17, wherein the acoustic windows are configured in shapes and sizes to deliver maximum energy deposition to the vessel wall therethrough.

21. The method of claim 20, wherein a relative placement of the acoustic windows and the inhibiting components of the stent-like structure are configured to provide areas of maximum energy transmission adjacent to areas of minimum or lessened energy transmission that create focal areas of energy deposition adjacent to focal areas of energy sparing to create structural instability within tissues affected by the acoustic pulses.

22. The method of claim 7, wherein the stent-like structure comprises configurations of one or more of: 1) an open cell stent pattern that minimizes the structural attenuating components of the stent-like structure and maximizes the non-attenuating open areas, 2) a scaffold of a material which transmits the energy without significant attenuation of the energy, 3) a scaffold comprising windows or apertures for the energy to be transmitted therethrough without attenuation, or 4) a pattern of non-attenuating windows within the stent-like structure oriented to provide maximum energy transmission therethrough and with purposeful attenuating means in other areas of the stent-like structure.

23. The method of claim 22, wherein the energy is applied to the vessel wall in a desired pattern that comprises both maximum and minimum energy delivery areas in the vessel wall adjacent to the windows and the attenuating components.

24. The method of claim 1, wherein the stent-like structure comprises configurations of: 1) an open cell stent pattern that minimizes the structural attenuating components of the stent-like structure and maximizes the non-attenuating open areas, 2) a scaffold of a material which transmits the energy without significant attenuation of the energy, 3) a scaffold comprising windows or apertures for the energy to be transmitted therethrough without attenuation, and 4) a pattern of non-attenuating windows within the stent-like structure oriented to provide maximum energy transmission therethrough and with purposeful attenuating means in other areas of the stent-like structure.

25. The method of claim 1, further comprising applying a drug or substance from a drug coated expandable element or the stent-like structure or the scoring elements of the stent-like structure.

26. The method of claim 1, wherein the gradual inflating of the expandable element is incremental and takes at least 90 seconds to inflate to the target size or a pressure of 5 atm.
